SEQUENCE LISTING (12 sequences)

SEQ ID NO: 1 - DNA, 90 bp, Homo sapiens
ccgggcccctgtgagcatcttaccggacagtgctggatttcccagcttgactctaacactgtctggtaacgatgttcaaaggtgacccgc

SEQ ID NO: 2 - DNA, 95 bp, Homo sapiens
ccagctcgggcagccgtggccatcttactgggcagcattggatggagtcaggtctctaatactgcctggtaatgatgacggcggagccctgcacg

SEQ ID NO: 3 - DNA, 68 bp, Homo sapiens
ccctcgtcttacccagcagtgtttgggtgcggttgggagtctctaatactgccgggtaatgatggagg

SEQ ID NO: 4 - DNA, 95 bp, Homo sapiens
cggccggccctgggtccatcttccagtacagtgttggatggtctaattgtgaagctcctaacactgtctggtaaagatggctcccgggtgggttc

SEQ ID NO: 5 - DNA, 83 bp, Homo sapiens
cgccggccgatgggcgtcttaccagacatggttagacctggccctctgtctaatactgtctggtaaaaccgtccatccgctgc

SEQ ID NO: 6 - DNA, 4261 bp, Homo sapiens
agccgccttcctatttccgcccggcgggcagcgctgcggggcgagtgccagcagagaggcgctcggtcctccctccgccctcccgcgccgggggcaggccctgcctagtctgcgtctttttcccccgcaccgcggcgccgctccgccactcgggcaccgcaggtagggcaggaggctggagagcctgctgcccgcccgcccgtaaaatggtcccctcggctggacagctcgccctgttcgctctgggtattgtgttggctgcgtgccaggccttggagaacagcacgtccccgctgagtgacccgcccgtggctgcagcagtggtgtcccattttaatgactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctgaagagcccagaggaggagtttggccaggtggactgtggcagatcaataaagaaaggcttcttcaggacagcactgccagagatgcctgggtgtgccacagaccttcctacttggcctgtaatcacctgtgcagccttttgtgggccttcaaaactctgtcaagaactccgtctgcttggggttattcagtgtgacctagagaagaaatcagcggaccacgatttcaagacttgttaaaaaagaactgcaaagagacggactcctgttcacctaggtgaggtgtgtgcagcagttggtgtctgagtccacatgtgtgcagttgtcttctgccagccatggattccaggctatatatttctttttaatgggccacctccccacaacagaattctgcccaacacaggagatttctatagttattgttttctgtcatttgcctactggggaagaaagtgaaggaggggaaactgtttaatatcacatgaagaccctagctttaagagaagctgtatcctctaaccacgagaccctcaaccagcccaacatcttccatggacacatgacattgaagaccatcccaagctatcgccacccttggagatgatgtcttatttattagatggataatggttttatttttaatctcttaagtcaatgtaaaaagtataaaaccccttcagacttctacattaatgatgtatgtgttgctgactgaaaagctatactgattagaaatgtctggcctcttcaagacagctaaggcttgggaaaagtcttccagggtgcggagatggaaccagaggctgggttactggtaggaataaaggtaggggttcagaaatggtgccattgaagccacaaagccggtaaatgcctcaatacgttctgggagaaaacttagcaaatccatcagcagggatctgtcccctctgttggggagagaggaagagtgtgtgtgtctacacaggataaacccaatacatattgtactgctcagtgattaaatgggttcacttcctcgtgagccctcggtaagtatgtttagaaatagaacattagccacgagccataggcatttcaggccaaatccatgaaagggggaccagtcatttattttccattttgttgcttggttggtttgttgctttatttttaaaaggagaagtttaactttgctatttattttcgagcactaggaaaactattccagtaatttttttttcctcatttccattcaggatgccggctttattaacaaaaactctaacaagtcacctccactatgtgggtcttcctttcccctcaagagaaggagcaattgttcccctgagcatctgggtccatctgacccatggggcctgcctgtgagaaacagtgggtcccttcaaatacatagtggatagctcatccctaggaattttcattaaaatttggaaacagagtaatgaagaaataatatataaactccttatgtgaggaaatgctactaatatctgaaaagtgaaagatttctatgtattaactcttaagtgcacctagcttattacatcgtgaaaggtacatttaaaatatgttaaattggcttgaaattttcagagaattttgtcttcccctaattcttcttccttggtctggaagaacaatttctatgaattttctctttatttttttttataattcagacaattctatgacccgtgtcttcatttttggcactcttatttaacaatgccacacctgaagcacttggatctgttcagagctgaccccctagcaacgtagttgacacagctccaggtttttaaattactaaaataagttcaagtttacatcccttgggccagatatgtgggttgaggcttgactgtagcatcctgcttagagaccaatcaacggacactggtttttagacctctatcaatcagtagttagcatccaagagactttgcagaggcgtaggaatgaggctggacagatggcggaagcagaggttccctgcgaagacttgagatttagtgtctgtgaatgttctagttcctaggtccagcaagtcacacctgccagtgccctcatccttatgcctgtaacacacatgcagtgagaggcctcacatatacgcctccctagaagtgccttccaagtcagtcctttggaaaccagcaggtctgaaaaagaggctgcatcaatgcaagcctggttggaccattgtccatgcctcaggatagaacagcctggcttatttggggatttttcttctagaaatcaaatgactgataagcattggatccctctgccatttaatggcaatggtagtctttggttagctgcaaaaatactccatttcaagttaaaaatgcatcttctaatccatctctgcaagctccctgtgtttccttgccctttagaaaatgaattgttcactacaattagagaatcatttaacatcctgacctggtaagctgccacacacctggcagtggggagcatcgctgtttccaatggctcaggagacaatgaaaagcccccatttaaaaaaataacaaacattttttaaaaggcctccaatactcttatggagcctggatttttcccactgctctacaggctgtgactttttttaagcatcctgacaggaaatgttttcttctacatggaaagatagacagcagccaaccctgatctggaagacagggccccggctggacacacgtggaaccaagccagggatgggctggccattgtgtccccgcaggagagatgggcagaatggccctagagttcttttccctgagaaaggagaaaaagatgggattgccactcacccacccacactggtaagggaggagaatttgtgcttctggagcttctcaagggattgtgttttgcaggtacagaaaactgcctgttatcttcaagccaggttttcgagggcacatgggtcaccagttgctttttcagtcaatttggccgggatggactaatgaggctctaacactgctcaggagacccctgccctctagttggttctgggctttgatctcttccaacctgcccagtcacagaaggaggaatgactcaaatgcccaaaaccaagaacacattgcagaagtaagacaaacatgtatatttttaaatgttctaacataagacctgttctctctagccattgatttaccaggctttctgaaagatctagtggttcacacagagagagagagagtactgaaaaagcaactcctcttcttagtcttaataatttactaaaatggtcaacttttcattatctttattataataaacctgatgcttttttttagaactccttactctgatgtctgtatatgttgcactgaaaaggttaatatttaatgttttaatttattttgtgtggtaagttaattttgatttctgtaatgtgttaatgtgattagcagttattttccttaatatctgaattatacttaaagagtagtgagcaatataagacgcaattgtgtttttcagtaatgtgcattgttattgagttgtactgtaccttatttggaaggatgaaggaatgaatctttttttcctaaa

SEQ ID NO: 7 - DNA, 4927 bp, Homo sapiens
cccggctttatatctatatatacacaggtatatgtgtatattttatataattgttctccgttcgttgatatcaaagacagttgaaggaaatgaattttgaaacttcacggtgtgccaccctacagtactgccctgacccttacatccagcgtttcgtagaaaccccagctcatttctcttggaaagaaagttattaccgatccaccatgtcccagagcacacagacaaatgaattcctcagtccagaggttttccagcatatctgggattttctggaacagcctatatgttcagttcagcccattgacttgaactttgtggatgaaccatcagaagatggtgcgacaaacaagattgagattagcatggactgtatccgcatgcaggactcggacctgagtgaccccatgtggccacagtacacgaacctggggctcctgaacagcatggaccagcagattcagaacggctcctcgtccaccagtccctataacacagaccacgcgcagaacagcgtcacggcgccctcgccctacgcacagcccagctccaccttcgatgctctctctccatcacccgccatcccctccaacaccgactacccaggcccgcacagtttcgacgtgtccttccagcagtcgagcaccgccaagtcggccacctggacgtattccactgaactgaagaaactctactgccaaattgcaaagacatgccccatccagatcaaggtgatgaccccacctcctcagggagctgttatccgcgccatgcctgtctacaaaaaagctgagcacgtcacggaggtggtgaagcggtgccccaaccatgagctgagccgtgaattcaacgagggacagattgcccctcctagtcatttgattcgagtagaggggaacagccatgcccagtatgtagaagatcccatcacaggaagacagagtgtgctggtaccttatgagccaccccaggttggcactgaattcacgacagtcttgtacaatttcatgtgtaacagcagttgtgttggagggatgaaccgccgtccaattttaatcattgttactctggaaaccagagatgggcaagtcctgggccgacgctgctttgaggcccggatctgtgcttgcccaggaagagacaggaaggcggatgaagatagcatcagaaagcagcaagtttcggacagtacaaagaacggtgatggtacgaagcgcccgtttcgtcagaacacacatggtatccagatgacatccatcaagaaacgaagatccccagatgatgaactgttatacttaccagtgaggggccgtgagacttatgaaatgctgttgaagatcaaagagtccctggaactcatgcagtaccttcctcagcacacaattgaaacgtacaggcaacagcaacagcagcagcaccagcacttacttcagaaacagacctcaatacagtctccatcttcatatggtaacagctccccacctctgaacaaaatgaacagcatgaacaagctgccttctgtgagccagcttatcaaccctcagcagcgcaacgccctcactcctacaaccattcctgatggcatgggagccaacattcccatgatgggcacccacatgccaatggctggagacatgaatggactcagccccacccaggcactccctcccccactctccatgccatccacctcccactgcacacccccacctccgtatcccacagattgcagcattgtcagtttcttagcgaggttgggctgttcatcatgtctggactatttcacgacccaggggctgaccaccatctatcagattgagcattactccatggatgatctggcaagtctgaaaatccctgagcaatttcgacatgcgatctggaagggcatcctggaccaccggcagctccacgaattctcctccccttctcatctcctgcggaccccaagcagtgcctctacagtcagtgtgggctccagtgagacccggggtgagcgtgttattgatgctgtgcgattcaccctccgccagaccatctctttcccaccccgagatgagtggaatgacttcaactttgacatggatgctcgccgcaataagcaacagcgcatcaaagaggagggggagtgagcctcaccatgtgagctcttcctatccctctcctaactgccagccccctaaaagcactcctgcttaatcttcaaagccttctccctagctcctccccttcctcttgtctgatttcttaggggaaggagaagtaagaggctacctcttacctaacatctgacctggcatctaattctgattctggctttaagccttcaaaactatagcttgcagaactgtagctgccatggctaggtagaagtgagcaaaaaagagttgggtgtctccttaagctgcagagatttctcattgacttttataaagcatgttcacccttatagtctaagactatatatataaatgtataaatatacagtatagatttttgggtggggggcattgagtattgtttaaaatgtaatttaaatgaaagaaaattgagttgcacttattgaccattttttaatttacttgttttggatggcttgtctatactccttcccttaaggggtatcatgtatggtgataggtatctagagcttaatgctacatgtgagtgacgatgatgtacagattctttcagttctttggattctaaatacatgccacatcaaacctttgagtagatccatttccattgcttattatgtaggtaagactgtagatatgtattcttttctcagtgttggtatattttatattactgacatttcttctagtgatgatggttcacgttggggtgatttaatccagttataagaagaagttcatgtccaaacgtcctctttagtttttggttgggaatgaggaaaattcttaaaaggcccatagcagccagttcaaaaacacccgacgtcatgtatttgagcatatcagtaacccccttaaatttaataccagataccttatcttacaatattgattgggaaaacatttgctgccattacagaggtattaaaactaaatttcactactagattgactaactcaaatacacatttgctactgttgtaagaattctgattgatttgattgggatgaatgccatctatctagttctaacagtgaagttttactgtctattaatattcagggtaaataggaatcattcagaaatgttgagtctgtactaaacagtaagatatctcaatgaaccataaattcaactttgtaaaaatcttttgaagcatagataatattgtttggtaaatgtttcttttgtttggtaaatgtttcttttaaagaccctcctattctataaaactctgcatgtagaggcttgtttacctttctctctctaaggtttacaataggagtggtgatttgaaaaatataaaattatgagattggttttcctgtggcataaattgcatcactgtatcattttcttttttaaccggtaagagtttcagtttgttggaaagtaactgtgagaacccagtttcccgtccatctcccttagggactacccatagacatgaaaggtccccacagagcaagagataagtctttcatggctgctgttgcttaaaccacttaaacgaagagttcccttgaaactttgggaaaacatgttaatgacaatattccagatctttcagaaatataacacatttttttgcatgcatgcaaatgagctctgaaatcttcccatgcattctggtcaagggctgtcattgcacataagcttccattttaattttaaagtgcaaaagggccagcgtggctctaaaaggtaatgtgtggattgcctctgaaaagtgtgtatatattttgtgtgaaattgcatactttgtattttgattattttttttttcttcttgggatagtgggatttccagaaccacacttgaaacctttttttatcgtttttgtattttcatgaaaataccatttagtaagaataccacatcaaataagaaataatgctacaattttaagaggggagggaagggaaagtttttttttattatttttttaaaattttgtatgttaaagagaatgagtccttgatttcaaagttttgttgtacttaaatggtaataagcactgtaaacttctgcaacaagcatgcagctttgcaaacccattaaggggaagaatgaaagctgttccttggtcctagtaagaagacaaactgcttcccttactttgctgagggtttgaataaacctaggacttccgagctatgtcagtactattcaggtaacactagggccttggaaattcctgtactgtgtctcatggatttggcactagccaaagcgaggcacccttactggcttacctcctcatggcagcctactctccttgagtgtatgagtagccagggtaaggggtaaaaggatagtaagcatagaaaccactagaaagtgggcttaatggagttcttgtggcctcagctcaatgcagttagctgaagaattgaaaagtttttgtttggagacgtttataaacagaaatggaaagcagagttttcattaaatccttttaccttttttttttcttggtaatcccctaaaataacagtatgtgggatattgaatgttaaagggatatttttttctattatttttataattgtacaaaattaagcaaatgttaaaagttttatatgctttattaatgttttcaaaaggtattatacatgtgatacattttttaagcttcagttgcttgtcttctggtactttctgttatgggcttttggggagccagaagccaatctacaatctctttttgtttgccaggacatgcaataaaatttaaaaaataaataaaaactaattaagaaattgaaaaaaaaaaaaaaaaaa

SEQ ID NO: 8 - DNA, 4815 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaaccat

SEQ ID NO: 9 - DNA, 6278 bp, Homo sapiens
tttctccctcccctctgggatgcgaaacgcgaggttttgtaacctttcctggcaattttagattttgtgtgggatttcctgtctagaagcagatacgaagatttttaagctgtttcaagatgtttccttccaatccataattatatttttaatatattcgagccatcattaaaatcactgctttcgtgattttaattattcaaataaacacttgcattttaaagacgtctgttgattataaacgaaaggtattttggtattctcattgtggagagatgacttgttatagcaaggagtggagcataggctattgcaattttaatttcctgttttagcgtcaaatagtgtgtgttccatattgagctgttgccgctgttgctgatgtggctttatgaaagttacaaattataatactgtggtagaaacaaattcagattcagatgatgaagacaaactgcatattgtggaagaagaaagtgttacagatgcagctgactgtgaaggtgtaccagaggatgacctgccaacagaccagacagtgttaccagggaggagcagtgaaagagaagggaatgctaagaactgctgggaggatgacacaggaaaggaagggcaagaaatcctggggcctgaagctcaggcagatgaagcaggatgtacagtaaaagatgatgaatgcgagtcagatgcagaaaatgagcaaaaccatgatcctaatgttgaagagtttctacaacaacaagacactgctgtcatttttcctgaggcacctgaagaggaccagaggcagggcacaccagaagccagtggtcatgatgaaaatggaacaccagatgcattttcacaattactcacctgtccatattgtgatagaggctataaacgctttacctctctgaaagaacacattaaatatcgtcatgaaaagaatgaagataactttagttgctccctgtgcagttacacctttgcatacagaacccaacttgaacgtcacatgacatcacataaatcaggaagagatcaaagacatgtgacgcagtctgggtgtaatcgtaaattcaaatgcactgagtgtggaaaagctttcaaatacaaacatcacctaaaagagcacttaagaattcacagtggagagaagccatatgaatgcccaaactgcaagaaacgcttttcccattctggctcctatagctcacacataagcagtaagaaatgtatcagcttgatacctgtgaatgggcgaccaagaacaggactcaagacatctcagtgttcttcaccgtctctttcagcatcaccaggcagtcccacacgaccacagatacggcaaaagatagagaataaaccccttcaagaacaactttctgttaaccaaattaaaactgaacctgtggattatgaattcaaacccatagtggttgcttcaggaatcaactgttcaacccctttacaaaatggggttttcactggtggtggcccattacaggcaaccagttctcctcagggcatggtgcaagctgttgttctgccaacagttggtttggtgtctcccataagtatcaatttaagtgatattcagaatgtacttaaagtggcggtagatggtaatgtaataaggcaagtgttggagaataatcaagccaatcttgcatccaaagaacaagaaacaatcaatgcttcacccatacaacaaggtggccattctgttatttcagccatcagtcttcctttggttgatcaagatggaacaaccaaaattatcatcaactacagtcttgagcagcctagccaacttcaagttgttcctcaaaatttaaaaaaagaaaatccagtcgctacaaacagttgtaaaagtgaaaagttaccagaagatcttactgttaagtctgagaaggacaaaagctttgaagggggggtgaatgatagcacttgtcttctgtgtgatgattgtccaggagatattaatgcacttccagaattaaagcactatgacctaaagcagcctactcagcctcctccactccctgcagcagaagctgagaagcctgagtcctctgtttcatcagctactggagatggcaatttgtctcctagtcagccacctttaaagaacctcttgtctctcctaaaagcatattatgctttgaatgcacaaccaagtgcagaagagctctcaaaaattgctgattcagtaaacctaccactggatgtagtaaaaaagtggtttgaaaagatgcaagctggacagatttcagtgcagtcttctgaaccatcttctcctgaaccaggcaaagtaaatatccctgccaagaacaatgatcagcctcaatctgcaaatgcaaatgaaccccaggacagcacagtaaatctacaaagtcctttgaagatgactaactccccagttttaccagtgggatcaaccaccaatggttccagaagtagtacaccatccccatcacctctaaacctttcctcatccagaaatacacagggttacttgtacacagctgagggtgcacaagaagagccacaagtagaacctcttgatctttcactaccaaagcaacagggagaattattagaaaggtcaactatcactagtgtttaccagaacagtgtttattctgtccaggaagaacccttgaacttgtcttgcgcaaaaaaggagccacaaaaggacagttgtgttacagactcagaaccagttgtaaatgtaatcccaccaagtgccaaccccataaatatcgctatacctacagtcactgcccagttacccacaatcgtggccattgctgaccagaacagtgttccatgcttaagagcgctagctgccaataagcaaacgattctgattccccaggtggcatacacctactcaactacggtcagccctgcagtccaagaaccacccttgaaagtgatccagccaaatggaaatcaggatgaaagacaagatactagctcagaaggagtatcaaatgtagaggatcagaatgactctgattctacaccgcccaaaaagaaaatgcggaagacagaaaatggaatgtatgcttgtgatttgtgtgacaagatattccaaaagagtagttcattattgagacataaatatgaacacacaggtaaaagacctcatgagtgtggaatctgtaaaaaggcatttaaacacaaacatcatttgattgaacacatgcgattacattctggagaaaagccctatcaatgtgacaaatgtggaaagcgcttctcacactctgggtcttattctcaacacatgaatcatcgctactcctactgtaagagagaagcggaagaacgtgacagcacagagcaggaagaggcagggcctgaaatcctctcgaatgagcacgtgggtgccagggcgtctccctcacagggcgactcggacgagagagagagtttgacaagggaagaggatgaagacagtgaaaaagaggaagaggaggaggataaagagatggaagaattgcaggaagaaaaagaatgtgaaaaaccacaaggggatgaggaagaggaggaggaggaggaagaagtggaagaagaagaggtagaagaggcagagaatgagggagaagaagcaaaaactgaaggtctgatgaaggatgacagggctgaaagtcaagcaagcagcttaggacaaaaagtaggcgagagtagtgagcaagtgtctgaagaaaagacaaatgaagcctaatcgtttttctagaaggaaaataaattctaattgataatgaatttcgttcaatattatccttgcttttcatggaaacacagtaacctgtatgctgtgattcctgttcactactgtgtaaagtaaaaactaaaaaaatacaaaatacaaaacacacacacacacacacacacacacacacacacacacacaaaataaatccgggtgtgcctgaacctcagacctagtaatttttcatgcagttttcaaagttaggaacaagtttgtaacatgcagcagattagaaaaccttaatgactcagagagcaacaatacaagaggttaaaggaagctgattaattagatatgcatctggcattgttttatcttatcagtattatcactcttatgttggtttattcttaagctgtacaattgggagaaattttataattttttattggtaaacatatgctaaatccgcttcagtattttattatgttttttaaaatgtgagaacttctgcactacaaaattcccttcacagagaagtataatgtagttccaacccgtgctaactaccttttataaattcagtctagaaggtagtaatttctaatatttagatgtcttagtagagcgtattatcatttaaagtgtattgttagccttaagaaagcagctgatagaagaactgaagtttcttactcacgtggtttaaaatggagttcaaaagattgccattgagttctgattgcagggactaacaatgttaatctgataaggacagcaaaatcatcagaatcagtgtttgtgattgtgtttgaatatgtggtaacatatgaaggatatgacatgaagctttgtatctcctttggccttaagcaagacctgtgtgctgtaagtgccatttctcagtattttcaaggctctaacccgccttcatccaatgtgtggcctacaataactagcatttgttgatttgtctcttgtatcaaaattcccaaataaaacttaaaaccactgactctgtcagagaaactgaaacactgggacatttcatccttcaattcctcggtattgattttatgttgattgattttcagaatttctctacagaaacgaaagggaaattttctaatctgctttatccatgtacttgcatttcagacatggacatgctattgttatttggctcataactgtttccaaatgttagttattatggacccaatttattaacaacattagctgatttttacctatcagtattattttatttcttttagtttatagatctgtgcaacatttttgtactgtatgtcttcaaacctggcagtattaatacccttcttactgacatatgtacttttagttttagaaaacttttatatttatgtgtcttatttttatatttctttatttattacacagtgtagtgtataatactgtagtttgtattaatacaataatatattttagtatgaaaatttggaaagttgataagatttaaagtagagatgcaattggttctcctgcattgagatttgatttaacagtgttatgttaacatttatacttgccttggactgtagaacagaacttaaatgggaatgtattagttttacaactacaatcaagtcattttacctttacccagtttttaatataaaacttaaattttgaaattcactgtgtgactaatagcatgatgctctgcagttttattaagaaatcagcctaaccatacaactctcatttccttagtaagccaaattaggattaacttctataaacagtgttgggaacaatgtttaacattttgtgccaatttgttcctgtattcatgtatgtaagttacagatctgactcttcatttttaagttccttgttacatcatggtcattttctagttttttaccagactcccatctcacaataaaatgcatcaacaagcctgaactgctgtcattcttttcatcattatcagtattttctttggaaaactgtgaaatggggtacattgtcatcctgcatttgattcatcttgagctgaatttgggtaacactaaatgttttagacattctccactaaattatggattttcttgtggctaaatgtttctggagaggtcagagttgacaaaacctcttcacaggttgctccttcttcctgaaatccttaatcctccgcatttcatgcttcaggtcatttcagggaagcctgggtttagatgcctttctgactctcagctcctgcacttctgtcatcatacctctgatactattatttatattccttccccactaggaacaggaaccacatttgtcatagtcactctcacattcctcactgcctaacagggtgcctggcataagttgggacaacagatatttgttgaataaaaatataatttgcatgtttatggagctcagctatgttctcactttttttgcttctaattccagaatatatgttaaatgatctaataatttgattattttcttataagtcttattaaacactagtcataatagacacaataaattatgccttctttttctattgccttaaaaaaaaaaa

SEQ ID NO: 10 - DNA, 9243 bp, Homo sapiens
atttcatttcttccactaaagcgtttgcggagacttcaaggtataatctatcccagatcctttcccagagagaaacttggcgatcacgttttcacatgatgctcacgctcagggcgcttcaattatccctccccacaaagataggtggcgcgtgtttcagggtctctcgtctctctcctacagaaaagaaaaagaaaaaaatgtcattagaagaggcgtaacacgtcagtccgtccccaggtttgtgtttcctggagtggccgaaagagatcagttctaacctgctctgcaggaataacggtcctgcctcccgacactcttggcgaggtttttgtacagtttgctccgggagctgtttcttcgcttccacctttttctcccccacacttcgcggcttcttcatgctttttcttctcaccatttctggccaaaactacaaacaagacttcgcagatcgagcctgcgtgctgccgaagcagggcgccgagtccatgcgaactgccatctgatccgctcttatcaatgaagcagccgatcatggcggatggcccccggtgcaagaggcgcaaacaagccaatcccaggaggaaaaacgtggtgaactatgacaatgtagtggacacaggttctgaaacagatgaggaagacaagcttcatattgctgaggatgacggtattgccaaccctctggaccaggagacgagtccagctagtgtgcccaaccatgagtcctccccacacgtgagccaagctctgttgccaagagaggaagaggaagatgaaataagggagggtggagtggaacacccctggcacaacaacgagattctacaagcctctgtagatggtccagaagaaatgaaggaagactatgacactatggggccagaagccacgatccagaccgcaattaacaatggtacagtgaagaatgcaaattgcacatcagattttgaggaatactttgccaaaagaaaactggaggaacgcgatggtcatgcagtcagcatcgaggagtaccttcagcgcagtgacacagccattatttacccagaagcccctgaggagctgtctcgccttggcacgccagaggccaatgggcaagaagaaaatgacctgccacctggaactccagatgcttttgcccaactgctgacctgcccctactgcgaccggggctacaagcgcttgacatcactgaaggagcacatcaagtaccgccacgagaagaatgaagagaacttttcctgccctctctgtagctacacgtttgcctaccgcacccagctcgagcggcatatggtgacacacaagccagggacagatcagcaccaaatgctaacccaaggagcaggtaatcgcaagttcaaatgcacagagtgtggcaaggccttcaaatataaacaccatctgaaagaacacctgcgaattcacagtggtgaaaaaccttacgagtgcccaaactgcaagaaacgtttctcccattctggttcctacagttcgcacatcagcagcaagaaatgtattggtttaatctctgtaaatggccgaatgagaaacaatatcaagacgggttcttcccctaattctgtttcttcttctcctactaattcagccattacccagttaagaaacaagttggagaatggaaaaccacttagtatgtctgaacagacaggcttacttaaaattaaaacagaaccactagacttcaatgactataaagttcttatggctacacacgggtttagtggcactagtccctttatgaatggtgggcttggagccaccagccctttaggagttcatccatctgctcagagtccaatgcagcacttaggtgtagggatggaagcccctttacttgggtttcccaccatgaatagtaatttaagtgaggtacaaaaggttctacagattgtggacaatactgtttccaggcaaaaaatggactgcaaggctgaagaaatttcaaagttgaaaggttatcacatgaaggatccatgctctcaacctgaggaacaaggagttacttctcctaatattccgcctgtcggtcttccggtagtgagtcataatggtgccactaaaagtattattgactatacgttggaaaaagtcaatgaagccaaagcttgcctccagagcttgactactgactcaaggagacagatcagtaatataaagaaagagaagctacgtactttaatagatttggtcactgatgacaaaatgattgagaaccacaacatatccactccattttcatgccagttctgtaaagaaagttttcctggccccatccctttgcatcagcatgaacgttacctttgtaagatgaatgaagagatcaaggcggtcctgcagcctcatgaaaacatagtccccaacaaagccggagtttttgttgataataaagccctcctcttgtcatctgtactttctgagaaaggaatgacaagccccatcaacccatacaaggaccacatgtctgtactcaaagcatactatgctatgaacatggagcccaactccgatgaactgctgaaaatttccattgctgtgggccttcctcaggaatttgtgaaggaatggtttgaacaacgaaaagtctaccagtactcaaattccaggtccccatccctggaaagaagctccaagccgttagctcccaacagtaaccctcccacaaaagactctttattacccaggtctcctgtaaaacctatggactccataacatcaccatctatagcagaactccacaacagtgttacgaattgtgatcctcctctcaggctaacaaaaccttcccattttaccaatattaaaccagttgaaaaattggaccactccaggagtaatactccttctcccttaaatctttcctccacatcttctaaaaactcccacagtagttcatacactccaaacagcttctcttctgaggagctccaggctgagcctttagacttgtcattaccaaaacaaatgaaagaacccaaaagtattatagccacaaagaacaaaacaaaagctagtagcatcagtttagatcataacagtgtttcttcctcatctgaaaactcagatgagcctctgaacttgacttttatcaagaaggaattttcaaattcaaataatctggacaacaaaagcactaacccagtgttcagcatgaacccatttagtgccaaacctttatacacagctcttccacctcaaagcgcatttccccctgctactttcatgccaccagtccagaccagtattcctgggctacgaccatacccaggactggatcagatgagcttcctaccacatatggcctacacctacccaactggagcagctacttttgctgatatgcagcaaaggagaaagtaccagcggaaacaaggatttcagggagaattgcttgatggagcacaagactacatgtcaggcctagatgatatgacagactccgactcctgtctgtctcgcaaaaagatcaagaagacagagagtggcatgtatgcatgtgacttatgtgacaagacattccagaaaagcagttcccttctgcgacataaatacgaacacacaggaaaaagaccacatcagtgtcagatttgtaagaaagcgtttaaacacaagcaccaccttatcgagcactcaaggcttcactcgggcgagaagccctatcagtgtgataaatgtggcaagcgcttctcacactcgggctcgtactcgcagcacatgaatcacaggtattcctactgcaagcgggaggcggaggagcgggaagcggcggagcgcgaggcgcgcgagaaagggcacttggaacccaccgagctgctgatgaaccgggcttacttgcagagcattacccctcaggggtactctgactcggaggagagggagagtatgccgagggatggcgagagcgagaaggagcacgagaaagaaggcgaggatggctacgggaagctgggcagacaggatggcgacgaggagttcgaggaggaagaggaagaaagtgaaaataaaagtatggatacggatcccgaaacgatacgagatgaagaagagactggagatcactccatggacgatagttcggaggatgggaaaatggaaaccaaatcagaccacgaggaagacaatatggaagatggcatgtaataaactactgcattttaagcttcctatttttttttccagtagtattgttacctgcttgaaaacactgctgtgttaagctgttcatgcacgtgcctgacgcttccaggaagctgtagagagggacagaaggggcggttcagccaagacagatgtagacggagttggagctgggtattgttaaaaactgcattatgcaaaaattttgtacagtgttaaggcctaaaaactgtgtggttcagagactaattcctgtgtttaatagcatttatactttaagcacaactagaaaattgtaagaattgcactctacttatgtatcactacaaactttaaaaaactatgtctaatttatattaatacattttaaaaaggtgcccgcactaccatacatcagtatttttattattattattgttattcctttttaatttaatgtgctcgcactacaatgcatcagtattatgattcctctgtactttcctttcgctattcatcaatttcccattttttttttcagcttaagtaaccacacaattttaggcctcaatttttttttttttctgtgaaggaacttgaagtgatgcatgtgtgaatttaagataccgaagtcttaaagtgacctggacgtgaaggaaaaagtaagatgagaaataaagaaagcctttgtaaggtggttttaaaagccttatatgcaaaccttttaatctgtgtttctgcaagtgccatccttgtacagtgttaagagggtaacatgggttacctttgcaccagcttcagtgttaagctcaccctgttctttgaagcacccatgtcagtattagaagaataggcagcagttccttagtttacatatgtttgtgcaattattttctgtacttttttgttcattaattttgtcagtattacaccaaactgtttttgcaacaaaaaaattttttttgcattcatttaattttaggtcaaataacattttatttatgtggctcattttatatttcctaattttatttatttcatactgtagtgtacagtattatagttcttcaatatatagatatattttagtaaaaaaggaacatgacgttgatcatttgggcaaattttacgtaaagagaagagcatttattgtgttttggaacattaattgtgagatgggatttttcaattttattattttatttttgtttttttccaattactggaaattccaaatttgggaacttttgatacgatcttgtgaaaacactgtattttcgactgaaaattccactttcttcatcttgttttttagctaaaaagagggactgttaaatacaatgtatgataccatgacaaaaatctttcctgaattgtctttgtaaaagtattattgaattttcaatttgtaatttcttttgaaaatgaccatgctcgaataaaaatgtagccaaactaagaatgtagttaatgagttctgtacttttagagagttttccttcaatgaccattaacatgtaacatgctttatgcttataataatgctaattatgtttttttcatataattttagtttagcaataattttgactggtaccaataactgttttttaaaattccatacctatgtacagcaattttacagcttttctcaactgatcctgattccagattgtgtatttttatgtgaggttatattattcaaatttagtctatttactttacagacatttctacttttgcattacgagtatttagagattatgtgttaaaaattcacttctctgtccaaggggtctttgtgatttattcaaaaaaaagtctaatttcaaaaagacagctattattcagtgttatttataatatgtaaccttttttaaaggattgggatagtttatctcactttttgaaatgcagacagtagtttaccgtttatctgaaactagaaggcgtgggtgggagaggaaaagctaaaagcaaatgctaacaaaaataaccgtgattttctaagacagtttttcagtttttacaagatgaccctaatattcagaatatgaatgtattcgtaggttttacataatgacttttatcaagaaactagattctgcttcttaaatctaattgccaagtgaagaataacagaaaaaacagattaccttatcaaatttacagctcttgaatatacagaactataatatagtagctgtccatgtattttttctactttagaatcaaagaagaaaagcatcattttgctattaaatttgctaaaattttgagtatgatatttccagttggcaagaacaacatatttatatttattccttagccataataccactttcctaaatttcacaaaagtcattctttgcaacttgaaactcaatagaaagtgtgtatgtgtgtgtgtgtatatatatatatatatacacacacacacatacacagaaaggatgtaatgaagatacagtaatagttgagcagacctttttagaaaaacatgtttttagctctatcttcaaactttctggcagagggggtggggggggcagggggaggagtggcatcaaaatgctatgcctcctgttatccacagcctagagtttttatatttggaaagtttagaaaattctatcctcgtttctccttctttgaatggcacaaataaatacactacataaatttttctggtttgaaaggctctaggcgataactttattaattcaacctgaaaatatcaagccattaaattttgtccgggtagaataaatccctgtggcctcttttaaagcaatgtaggtctctgttgcccatggggcatatctgtgtcccaatccacaagagataggaccaacaaacaatgaatgtgcaacctaactctttctccttggaaagaagaaagtgtgcacgaagtagaggagggtgggcagaccctgccttgcccctcctgttacccccttctctgtcatttgttcctaactccatttcataggcaggctcagaatacctgagtctgaaaatatcaggataacacttgtgaattgtgacaatcactacaatgtcccatatctgaggagttttttttaatgctatttatccgctggacacgattgcacattagggctgcataatcctctaactctagggaaaaataaaaacttttgatttgtcttaagattcttctccaaggtcgcaaacaagaaattcccctccacaaccaagagatgtgcattttagtaacatcagatgtgttcttctgttttatcaactacttactcttcccacacgcttagttctaaatctaacctttcccccctcgaatagggggcaggggaggatgaggaaacactggaacaactgaacacccctgcccattttctccaagagccttttgtattctagcatatctgtgcaatcttttcttttttcttcacatgacactgtaagcttaggcctgaaataactgggaagagagatgcgtatcagaatttctccgcaagagctaaacaaaacatacatcttccttagcatgaattggactgggggcggagtgggagggcttggaggaaaggggaaagaagggactatatttgaataaatatgaataaatgtattagatacttttcacaatcagataacttttaaaaaggtcattttttatctttctaataatgtaagccttaataaaagcaaatcttagtcacaaatttgaggagactgcccaataataagtttacatgtatttgaactgaaaaattgttaaccatgcttttgctccaagatgtgtgaggccattcaggggctgtagggccctggatatacacacaaacaagtgtgtgtatatctggagccccacacattgtaataaacacagctgcatttatttgactatgtgatcccatgtacatgtaaaaacattcaaacaaacacactcagcggatttatttattgtgcaatggggcaattattcaaataaacatgctcaatgcaattatttgaatctcacattgcatgttcatcaatcatagcactaaaaaaagagggggaaaaaacaccaaagaattcacatggggaaaaaatatatatatgaaaaccaccttattatagattttatagggcagctgaggttatggctcccttcttaactgtaactcaactattctgtattcaatgacatttgtttctaatgattaattggttcactcacttgatcatataatagcaaactttataaacctgtattgtgtagagatgtgaaatctctatatttcaagagcagaagagttctttctagacaccttacatcaagggacactggtccaattattatcgcttatataagcactcctataaattctgaaaaattttatacatgcaacaaaacattcctacatttgaagacattaagaaaaatcacaggtgactcatctgatcattctatatattaataaatattatgacatatatgtgaacacatcacaaatcatattggtgtaccaagaggcaatttatgcctctcttaagtatgtactgacataacctaatatactaaaatgggaaggggcttttagtcactgaaatatgcatcgtgtaacaaagatgaagaaaatacatggcttgtgcccatcataaaaaaagattcagactgaaggcttagctttggttttttcaattaaattgttaaactgtgcacagtgatttttttttagaacttgagacatttgtgatgttggctgtttaaatctttgttaccttcgctgtgaattgaaattgtacatatttagtaaatcatgcagacaaaacaaactttttagacaatatttttattggagagttttcttttcctgtatccatgttaaaaaaaaaaaagacctcctttcccaaaataaaaatgtcaatactaaatttaaagaagtataaaggaatgattgcttcctttagagcaaaatatttaaataaacatggagataattggcaacatgttctttttgggctagtaggctgtgtccaattttttgggtctgatgtttcagagggcctctgtttcagggttgaagatgatatattaatctcggaattaaacaaatgctattaaataac

SEQ ID NO: 11 - DNA, 6493 bp, Homo sapiens
gccatgtttcaatctggccccagtggctttttctctgaaagcaaacgtgtgtcttttacaccagggctttctccccaccccagggggtgtcttccatccttttgtggctcagttgaaggcgaaaagggctccaaaccactaactaaccagaggagagccccttcttccacctccagggagaatttcagatttaatttgtccgaagatagcgtgctctcttcttactcatttgccatcattacgaggaaaacaaaccaccaccttggcttcaagatcctgggtagaggctcacggtcttttcaaccatctttggcgaggccttgcttccttccactcgaggtatgttctgtcttgtgctttttcttttagaagctactaaagggtgttggggatgcttctgactattatgaaggccaaaaggcctgttgactggggctgcttttaaccctttcctatttgctgagaatgcagccgtgtgacagtaactg[450 bp omitted]cgatctccagtgctgaagagtttgatgacgacgagaagattgaggtggatgacccccctgacaaggaggacatgcgatcaagcttcaggtcgaatgtgttgacggggtcggctccccagcaggactacgataagctgaaggcactcggaggggaaaactccagcaaaactggactctctacgtcaggcaatgtggagaaaaacaaagctgttaagagagaaacagaagccagttctataaacctgagtgtttatgaaccttttaaagtcagaaaagcagaggataaattgaaggaaagctctgacaaggtgctggaaaacagagtcctagatgggaagctgagctccgagaagaatgacaccagcctccccagcgttgcgccatcaaagacaaagtcgtcctccaagctctcgtcctgcatcgctgccatcgcggctctcagcgctaaaaaggcggcttcagactcctgcaaagaaccagtggccaattcgagggaatcctccccgttaccaaaagaagtaaatgacagtccgagagccgctgacaagtctcctgaatcccagaatctcatcgacgggaccaaaaaaccatccctgaagcaaccggatagtcccagaagcatctcaagtgagaacagcagcaaaggatccccgtcctctcccgcagggtccacaccagcaatccccaaagtccgcataaaaaccattaagacatcttctggggaaatcaagagaacagtgaccagggtattgccagaagtggatcttgactctggaaagaaaccttccgagcagacagcgtccgtgatggcctctgtgacatcccttctgtcgtctccagcatcagccgccgtcctttcctctccccccagggcgcctctccagtctgcggtcgtgaccaatgcagtttcccctgcagagctcacccccaaacaggtcacaatcaagcctgtggctactgctttcctcccagtgtctgctgtgaagacggcaggatcccaagtcattaatttgaagctcgctaacaacaccacggtgaaagccacggtcatatctgctgcctctgtccagagtgccagcagcgccatcattaaagctgccaacgccatccagcagcaaactgtcgtggtgccggcatccagcctggccaatgccaaactcgtgccaaagactgtgcaccttgccaaccttaaccttttgcctcagggtgcccaggccacctctgaactccgccaagtgctaaccaaacctcagcaacaaataaagcaggcaataatcaatgcagcagcctcgcaaccccccaaaaaggtgtctcgagtccaggtggtgtcgtccttgcagagttctgtggtggaagctttcaacaaggtgctgagcagtgtcaatccagtccctgtttacatcccaaacctcagtcctcccgccaatgcagggatcacgttaccgacgcgtgggtacaagtgcttggagtgtggggactcctttgcacttgaaaagagtctgacccagcactacgacagacggagcgtgcgcatcgaagtaacgtgcaaccattgtacaaagaacctcgttttttacaacaaatgcagcctcctttcccatgcccgtgggcataaggagaaaggggtggtaatgcaatgctcccacttaattttaaagccagtcccagcagatcaaatgatagtttctccgtcaagcaatacttccacttcaacttccactcttcagagccctgtgggagctggcacacacactgtcacaaaaattcagtctggcataactgggacagtcatatcggctccttcaagcactcccatcaccccagccatgcccctagatgaagacccctccaaactgtgtagacatagtctaaaatgtttggagtgtaatgaagtcttccaggacgagacatcactggctacacatttccagcaggctgcagatacgagtggacaaaagacttgcactatctgccagatgctgcttcctaaccagtgcagttatgcatcacaccagagaatccatcagcacaaatctccctacacctgccctgagtgtggggccatctgcaggtcggtgcacttccagacccacgtcaccaagaactgtctgcactacacgaggagagttggttttcgatgtgtgcattgcaatgttgtgtactctgatgtggctgctctgaagtctcacattcaaggttctcactgtgaagtcttctacaagtgtcctatttgtccaatggcgtttaagtctgccccaagcacacattcccacgcctacacacagcatcctggcatcaagataggagaaccaaaaataatatataagtgttccatgtgcgacactgtgttcaccctgcaaaccttgctgtatcgccactttgaccaacacattgaaaaccagaaggtgtctgttttcaagtgtccagactgttctcttttatatgcacagaagcaacttatgatggaccatatcaagtctatgcatggaacattgaaaagtattgaagggcctccaaacttgggtataaacttgcctttgagcattaagcctgcaactcaaaattcagcaaatcagaacaaagaggacaccaaatccatgaatgggaaagagaaattggaaaagaaatctccatctcctgtgaaaaaatcaatggaaaccaagaaagtggccagtcctgggtggacgtgttgggagtgtgactgcctgttcatgcagagagatgtgtacatatcccacgtgaggaaggagcacgggaagcaaatgaagaaacacccctgccgccagtgtgacaagtctttcagctcgtcccacagcctgtgccggcacaaccggatcaagcacaaaggcatcaggaaagtgtacgcctgctcgcactgcccagactccagacgtacctttaccaaacgtttgatgctggagaagcacgtccagctgatgcatggcatcaaggaccctgacctgaaagaaatgacagatgccaccaatgaggaggaaacagaaataaaagaagacactaaggtccccagtcccaagcggaagttggaagaaccagttctggagttcaggcctccccgaggagcaatcactcaaccactgaaaaagctgaaaatcaatgtttttaaggttcacaagtgtgccgtgtgtggcttcaccaccgaaaacctgctgcaattccacgaacacatccctcagcacaaatcggatggttcttcctaccagtgccgggagtgtggcctctgctacacgtctcacgtctctctgtccaggcacctcttcatcgtacacaagttaaaggaacctcagccagtgtccaagcaaaatggggctggggaagataaccaacaggagaacaaacccagccacgaggatgaatcccctgatggcgccgtgtcagacagaaagtgcaaagtgtgcgcaaaaacttttgaaactgaagctgccttaaatactcacatgcggacacacggcatggccttcatcaaatccaaaaggatgagctcagccgagaaatagccacagatgctccatgaggaaaatccctgtccacattggaataaaaaagacatttttgttacaaagtttgcagtataatagagttaacagtactgtctaggctgttgcaatatattctctttcaatgtaccttccttcacctcgtcgtatatatcctcgataagtattaaaacagtatttgagtttaaaagagtttgtatatatttaaatgaataactttttatactctttgttacatgtttgtatcagtatttagtggaaaaccatttgagttgttttgggttagaatttttctttttgtactgtttctttaaaacagagttcttagtaacaggggcagttcctgaattcaaataaaccattttgtatgtttggattttgaatgggttaactaattacaggctaaaataatgccttttttagtgtttttaatttttagaattcactacataaattgtaagtaattgtgggtctcaaaaacactaggaacttttaagtgtcttagcacttcctcgatgtgcctgccctgagggagtgagttcacatttgagacaactgcactccagtgtggacgtgcctttgtcttcaggccatgccgaagggtgtttaaagcagtcttgcaggtcgctcctttcccagccgtggataaaaactgaagctaggaatctaataaggaatgctgatttcctcagttccattttgaggaatggggaaggctattctaaagaaaaaaatgggatttgttttctcggcagatctgcaaggctggctttaagagcacaaggagggaaagtaacgaaagggctggactactataaaagttacaaatacgtagttagaccaatagatttatatagtcaggtttttgtcatgtaatttattaactaactattacagaaacacagctaagaatatcaagtatttctctggctcttgacagaaaaaaatcagttgacttaaccctttgctgtcaaaagagttggcgtttcctgttctgggtgctactgccaaacgttatggtacttagagtcgggatgcacaacttcaaccaccgacttatcaatgcagccgcctgtgtattgcaattggccgttaccttaagcactgagccacccgggtttagttcagccatttcaagaagtatatttaacgtcggtagttctgctttattaaaatgcagcagaggtactcttctgtcccttccgtttatagttctctgagagagttctattttttggttttgttttgtgttttcttttgcattttgtatcttgtatttatccctgaacatgttttgtaccttttttttttttttttttaagaaaaggaattcttttgtgtatatatagatacttgcatgatatactgtagtcaatgttcggttcctcaaaaggtcttgctgctgtcaggtgttatgcactccatccatcataactgtatgaaacacatttcatatgtaaataaacgtgggacatttggcccttgtgcttctgtgagagaattattgatggtgggtctctgacatctttgtgaagtttgggaagtaattaattgcagcgacaagctacagggtgttgcagaattcttcccactcagaagaatggcatattcgttctcattagtaatcagctattttgtcactttcttgttgactccatcagtacatgggtacaatccgagggtgtgaatttcagcttgaaattccattgctgttccttgttttgtttgtattgctctaagttgtattcataatagcactttcatatgtttctgcatttgaaccttgcaataagcctgtgtggtaggccacataggtccgaataacctagttttacagttgagggagctgagctcagattcagttctttgccgaagccctcatagctggtaagtggctttgcatattagaacccaaatattttgctctctaaatctaatgctcgctctatgtggttatgtacatattgacaaatattcatttattcaacaaataaaaagtatgtacaaaacaaaaaaaaaaaaaaaaaaaaaaaaaaaaaa

SEQ ID NO: 12 - DNA, 3144 bp, Homo sapiens
agaggcggcggcggcagccgcggcgacggcggtccggtgcgaggcagagtgctagcgggagcgcgagccagcaagaggcgcctgcgcgatgtccgggcccctgagcccgcggcgctgagccagccgggacggacatgcgcgggagggcgccgcggggcagccgccgctcctccgggggaatgaaagctactggttgattttaaagtgcctgggcctcacaggtttggagatgtcccagaataaggcacaatgtcaatagcaggagttgctgctcaggagatcagagtcccattaaaaactggatttctacataatggccgagccatggggaatatgaggaagacctactggagcagtcgcagtgagtttaaaaacaactttttaaatattgacccgataaccatggcctacagtctgaactcttctgctcaggagcgcctaataccacttgggcatgcttccaaatctgctccgatgaatggccactgctttgcagaaaatggtccatctcaaaagtccagcttgccccctcttcttattcccccaagtgaaaacttgggaccacatgaagaggatcaagttgtatgtggttttaagaaactcacagtgaatggggtttgtgcttccacccctccactgacacccataaaaaactccccttcccttttcccctgtgcccctctttgtgaacggggttctaggcctcttccaccgttgccaatctctgaagccctctctctggatgacacagactgtgaggtggaattcctaactagctcagatacagacttccttttagaagactctacactttctgatttcaaatatgatgttcctggcaggcgaagcttccgtgggtgtggacaaatcaactatgcatattttgataccccagctgtttctgcagcagatctcagctatgtgtctgaccaaaatggaggtgtcccagatccaaatcctcctccacctcagacccaccgaagattaagaaggtctcattcgggaccagctggctcctttaacaagccagccataaggatatccaactgttgtatacacagagcttctcctaactccgatgaagacaaacctgaggttccccccagagttcccatacctcctagaccagtaaagccagattatagaagatggtcagcagaagttacttcgagcacctatagtgatgaagacaggcctcccaaagtaccgccaagagaacctttgtcaccgagtaactcgcgcacaccgagtcccaaaagccttccgtcttacctcaatggggtcatgcccccgacacagagctttgcccctgatcccaagtatgtcagcagcaaagcactgcaaagacagaacagcgaaggatctgccagtaaggttccttgcattctgcccattattgaaaatgggaagaaggttagttcaacacattattacctactacctgaacgaccaccatacctggacaaatatgaaaaattttttagggaagcagaagaaacaaatggaggcgcccaaatccagccattacctgctgactgcggtatatcttcagccacagaaaagccagactcaaaaacaaaaatggatctgggtggccacgtgaagcgtaaacatttatcctatgtggtttctccttagaccttggggtcatggttcagcagaggttacataggagcaaatggttctcaattttccagtttgattgaagtgcagagaaaaatcccttagattgcaaaataaaatagttgaactctctgtcttcatgtggaaggtttagagcagttgtgagatgctgttatgctgagaaaccctgactttgttagtgttggaaaaaagtcttacaagtctataatttaaagatgtgatggtggggaggggaggatggggaagctttttatatatgcatacattacatacctatatataaacttgtggtataaccatagaccatagctgcaggttaaccaattagttactatcgtagagtaatatatattcagaataataaactcaagctggagaaatgagtcctgatagactgaaaattgagcaaatggaagaagatacagtattgtttagatcagaatcattaaaaaatatttttgtttagtaagtttgaagatttctggcttttaggccttttctattttgttccatttatttttgcaggcaatcttttccatggagggcagggtatccattctttaccatgggtgtacctgcttaggttaaaaatcataccaaggcctcatacttccaggtttcatgttgcgtcttgttgagggagggagagcaggttacttggcaaccatattgtcacctgtacctgtcacacatcttgaaaaataaaacgataatagaactagtgactaattttcccttacagttcctgcttggtcccacccactgaagtagctcatcgtagtgcgggccgtattagaggcagtggggtacgttagactcagatggaaaagtattctaggtgccagtgttaggatgtcagttttacaaaataatgaagcaattagctatgtgattgagagttattgtttggggatgtgtgttgtggttttgctttttttttttagactgtattaataaacatacaacacaagctggccttgtgttgctggttcctattcagtatttcctggggattgtttgctttttaagtaaaacacttctgacccatagctcagtatgtctgaattccagaggtcacatcagcatctttctgctttgaaaactctcacagctgtggctgcttcacttagatgcagtgagacacatagttggtgttccgattttcacatccttccatgtatttatcttgaagagataagcacagaagagaaggtgctcactaacagaggtacattactgcaatgttctcttaacagttaaacaagctgtttacagtttaaactgctgaatattatttgagctatttaaagcttattatattttagtatgaactaaatgaaggttaaaacatgcttaagaaaaatgcactgatttctgcattatgtgtacagtattggacaaaggattttattcattttgttgcattattttgaatattgtcttttcattttaataaagttataatacttatttatgataccattaaaaaaaaaaaaaaaa

(19) United States
(12) Patent Application Publication
(10) Pub. No.: US 2013/0058925 A1
(43) Pub. Date: Mar. 7, 2013

(54) EPITHELIAL BIOMARKERS FOR CANCER PROGNOSIS

(71) Applicants: Colin P. Dinney, Houston, TX (US); Alexandru George Floares, Cluj-Napaca (RO); Liana Adam, Pearland, TX (US)

(72) Inventors: Colin P. Dinney, Houston, TX (US); Alexandru George Floares, Cluj-Napaca (RO); Liana Adam, Pearland, TX (US)

(73) Assignees: BOARD OF REGENTS, THE UNIVERSITY OF TEXAS SYSTEM, Austin, TX (US); Alexandru George FLOARES, Cluj-Napca (RO)

(21) Appl. No.: 13/580,606

(22) PCT Filed: Feb. 25, 2011
(86) PCT No.: PCT/US2011/026329

§ 371 Date: Nov. 7, 2012

Related U.S. Application Data
(60) Provisional application No. 61/308,601, filed on Feb. 26, 2010.

(51) Int. Cl. G01N 21/64; G01N 21/75; A61P 35/00; G01N 33/566; A61K 39/395; C40B 30/04; G01N 21/76

(52) U.S. Cl. 424/133.1; 506/9; 435/6.12; 435/7.1; 435/7.92; 435/6.11; 436/501

(57) ABSTRACT

Methods, systems and compositions for the prognosis and classification of cancer, especially bladder cancer, are provided. For example, in certain aspects methods for cancer prognosis using expression analysis of selected biomarkers such as miR-200 and TGFalpha are described.

This application claims the benefit of U.S. Provisional Patent Application No. 61/308,601, filed Feb. 26, 2010, the entirety of which is incorporated herein by reference.

The sequence listing that is contained in the file named “UTFCP1050WO_ST25.txt”, which is 56.0 KB (as measured in Microsoft Windows®) and was created on Feb. 25, 2011, is filed herewith by electronic submission and is incorporated by reference herein.

### BACKGROUND OF THE INVENTION

1. Field of the Invention

The present invention relates generally to the fields of oncology, molecular biology, cell biology, and cancer. More particularly, it concerns cancer prognosis or treatment based on the determination of molecular marker-based phenotypes.

2. Description of Related Art

Gene expression profiling studies of various cancers have discovered consistent gene expression patterns associated with pathological or clinical phenotype, elucidating subtypes of cancer previously unidentified with conventional technologies. This new technology has been used successfully to predict clinical outcomes and survival rates and to identify potential therapeutic targets and prognostic marker genes. Better understanding of the fundamental biology of these genes may not only improve prognostication but also offer new individualized therapeutic options.

However, despite many attempts to establish pre-treatment prognostic markers to understand the clinical biology of cancer patients, validated clinical or biomarker parameters are lacking in many aspects. Therefore, there remains a need to discover novel prognostic markers for cancer patients, especially bladder cancer patients.

### SUMMARY OF THE INVENTION

The present invention overcomes major deficiencies in the art by providing a method for obtaining prognostic information of a subject determined to have a cancer, comprising determining whether the subject's cancer has an epithelial phenotype, wherein the epithelial phenotype is determined by an expression profile comprising two or more of: a) a higher expression level of tumor growth factor (TGF)-alpha as compared to a reference level thereof; b) a higher expression level of one or more miR-200 family members as compared to a reference level thereof; c) a lower expression level of one or more miR-200 family targets as compared to a reference level thereof; d) a higher expression level of p63 as compared to a reference level thereof; and e) a higher expression level of CDH-1 as compared to a reference level thereof; wherein such an epithelial phenotype indicates a poor prognosis. In a particular aspect, the epithelial phenotype may be determined by an expression profile comprising a) and b) to achieve an optimal prognosis. In a further aspect, the epithelial phenotype may be determined by an expression profile comprising three, four, or all of a)-e). The subject may be a human.

In some other aspects, there may also comprise prognosis methods that if the subject's cancer has: a) an expression level of tumor growth factor (TGF)-alpha not higher than a reference level thereof; b) an expression level of one or more miR-200 family members not higher than a reference level thereof; c) an expression level of one or more miR-200 family targets not lower than a reference level thereof; d) an expression level of p63 not higher than a reference level thereof; and/or e) an higher expression level of CDH-1 not higher than a reference level thereof; then such is indicative of a favorable prognosis.

In a particular aspect, miR-200 family members may be miR-200b, mir-200c, miR-205, miR-429 and/or miR-141. Examples of miR-200 family targets include, but are not limited to, Zinc finger E-box binding homeobox 1 (Zeb1), Zinc finger E-box binding homeobox 2 (Zeb2), Zinc figure protein 532 (ZNF532) a-d, ZNF532a&b, and/or ERBB receptor feedback inhibitor 1 (ERRFI-1). The ZNE532a-d may be a biomarker identified by a probe or primer specific for a sequence that is common for all four isoforms of ZNF532 gene, whereas the ZNF532a&b may be a biomarker identified by a probe or a primer specific for the sequence common for isoforms ZNF532a and ZNF532b, but not ZNF532c and ZNF532d.

To improve accuracy of prognosis, certain aspects of the invention may further comprise using a predictive analytic to generate a prognosis. The predictive analytic may be a method, a system, or a tangible computer program product using neural networks, support vector machines, decision trees, classification and regression trees (CART), or genetic programming. In a particular aspect, the predictive analytic may be a CART-based system or a CART method.

Based on the non-linear relationship between the biomarkers, there may be a method comprising a set of rules for cancer prognosis using the expression information of the biomarkers. For example, the predictive analytic may comprise one or more rules of: i) if the subject's cancer has a miR-200b expression level not higher than a reference level thereof, then such is indicative of a favorable prognosis; ii) if the subject's cancer has a miR-200b expression level higher than a reference level thereof, a TGF-alpha expression level not higher than a reference level thereof, a CDH-1 expression level not higher than a reference level thereof, then such is indicative of a favorable prognosis; iii) if the subject's cancer has a miR-200b expression level higher than a reference level thereof, a TGF-alpha expression level not higher than a reference level thereof, a CDH-1 expression level higher than a reference level thereof, and a ZNF-532 expression level lower than a reference level thereof, then such is indicative of a poor prognosis; iv) if the subject's cancer has a miR-200b expression level higher than a reference level thereof, a TGF-alpha expression level not higher than a reference level thereof, a CDH-1 expression level higher than a reference level thereof, and a ZNF-532 expression level not lower than a reference level thereof, then such is indicative of a favorable prognosis; v) if the subject's cancer has a miR-200b expression level higher than a reference level thereof, a TGF-alpha expression level higher than a reference level thereof, a ZEB1 expression level lower than a reference level thereof, then such is indicative of a poor prognosis; and vi) if the subject's cancer has a miR-200b expression level higher than a reference level thereof, a TGF-alpha expression level higher than a reference level thereof, a ZEB1 expression level not lower than a reference level thereof, then such is indicative of a favorable prognosis. In a particular aspect, the method may comprise two, three, four, five, or all of the rules i)-vi) (see, e.g., FIG. 7).

In further aspects, the method may comprise obtaining a sample of the subject's cancer. For assessing biomarker expression, the sample may be serum, saliva, biopsy or needle aspirate, which may be paraffin-embedded or frozen. The method may further comprise isolation nucleic acid of the subject's cancer. In particular aspects, the method may comprise testing mRNA expression or protein expression of the subject's cancer, in particular one or more of the biomarkers described above. In an alternative aspect, the method may comprise analyze a predetermined expression profile. The predetermined expression profile may be obtained from a lab, a service provider, or a technician.

The cancer for prognosis or classification with certain aspects of the present methods may be oral cancer, oropharyngeal cancer, nasopharyngeal cancer, respiratory cancer, urogenital cancer, gastrointestinal cancer, central or peripheral nervous system tissue cancer, an endocrine or neuroendocrine cancer or hematopoietic cancer, glioma, sarcoma, carcinoma, lymphoma, melanoma, fibroma, meningioma, brain cancer, oropharyngeal cancer, nasopharyngeal cancer, renal cancer, biliary cancer, pheochromocytoma, pancreatic islet cell cancer, Li-Fraumeni tumors, thyroid cancer, parathyroid cancer, pituitary tumors, adrenal gland tumors, osteogenic sarcoma tumors, multiple neuroendocrine type I and type II tumors, breast cancer, lung cancer, head and neck cancer, prostate cancer, esophageal cancer, tracheal cancer, liver cancer, bladder cancer, stomach cancer, pancreatic cancer, ovarian cancer, uterine cancer, cervical cancer, testicular cancer, colon cancer, rectal cancer or skin cancer. Particularly, the cancer is an epithelial cancer, such as bladder cancer.

The skilled artisan will understand that any methods known in the art for assessing gene expression can be used in the present methods and compositions. The testing to assess gene expression may comprise RNA quantification, such as obtaining RNA of the sample, reverse transcription, amplification and/or probe hybridization. The techniques that may be used in the testing for RNA quantification may include, but not limited to, cDNA microarray, quantitative RT-PCR, in situ hybridization, Northern blotting, nuclease protection, a chip-based expression platform, invader RNA assay platform or b-DNA, detection platform, or a combination thereof. In particular, cDNA microarray may be used for its high-throughput and high efficiency. Quantitative RT-PCR may also be used alone or in combination with other quantification methods for validation or confirmation.

Alternatively, the testing may comprise antibody detection for expression at a protein level, such as immunohistochemistry, an enzyme-linked immunosorbent assay (ELISA), a radioimmunoassay (RIA), an immunoradiometric assay, a fluoroimmunoassay, a chemiluminescent assay, a bioluminescent assay, a gel electrophoresis, a Western blot analysis, an expression array, or a combination thereof.

In a further aspect, the method may comprise recording the prognostic information in a tangible medium. For example, such a tangible medium may be a computer-readable medium, such as a computer-readable disk, a solid state memory device, an optical storage device or the like, more specifically, a storage device such as a hard drive, a Compact Disk (CD) drive, a floppy disk drive, a tape drive, a random access memory (RAM), etc.

In certain aspects of the invention, the poor prognosis may indicate high risk of recurrence, poor survival, higher chance of cancer progress or metastasis, or a low response to or a poor clinical outcome after a conventional therapy such as surgery, chemotherapy and/or radiation therapy. In an other aspect, the good prognosis may comprise low risk of recurrence, good survival, lower chance of cancer progress or metastasis, or a high response to or a good clinical outcome after a conventional therapy.

Based on the prognosis determination, the methods may comprise reporting the prognosis to the subject, a health care payer, a physician, an insurance agent, or an electronic system. In further aspects, the methods may comprise prescribing or administering a treatment to the subject: for example, such a treatment would be a conventional therapy like surgery, chemotherapy and/or radiation therapy to the subject if good prognosis is identified, or an alternative treatment other than surgery, chemotherapy and radiation therapy to the subject if poor prognosis is identified.

In a certain aspect, there may be also provided a method comprises treating a cancer patient with a determined expression profile comprising one or more of the biomarkers including: a) TGF-alpha; b) one or more miR-200 family members; c) one or more miR-200 family targets; d) p63; and e) E-cadherin. For example, the cancer patient is a bladder cancer patient.

In a further aspect, there may also be provided a method of developing a treatment plan for a subject determined to have a cancer comprising: a) determining whether the subject's cancer has an epithelial phenotype, wherein if the subject's cancer has an epithelial phenotype, the subject is more likely to exhibit a poor response to one or more conventional cancer therapy and/or a favorable response to an alternative therapy such as an epidermal growth factor receptor (EGFR)-directed therapy; and b) developing the treatment plan. For example, the one or more conventional cancer therapy comprise chemotherapy, radiation therapy, and/or surgery. The method may further comprise treating the subject with EGFR-directed therapy if the subject's cancer is determined to have an epithelial phenotype. Alternatively, the method may comprise treating the subject with one or more conventional cancer therapy if the subject's cancer is determined not to have an epithelial phenotype.

Furthermore, in certain aspects of the invention, there is also provided a kit comprising a plurality of antibodies that bind to one or more biomarker proteins; or probes or primers that bind to one or more biomarker gene sequences to assess expression of the biomarkers in cells. In a particular aspect, the kit is housed in a container. For example, the biomarkers may include a) TGF-alpha; b) one or more miR-200 family members; c) one or more miR-200 family targets; d) p63; and/or e) E-eadherin.

In a further aspect, the kit may also comprise instructions to indicate that a subject has a poor prognosis if a cancer sample from the subject has an epithelial phenotype as determined above; or to indicate that a subject has a good prognosis if the sample does not have such an epithelial phenotype.

In certain aspects, there may also be provided a tangible, computer-readable medium comprising an expression profile of a cancer patient, wherein the expression profile exhibits expression level of two or more of: a) TGF-alpha; b) one or more miR-200 family members; c) one or more miR-200 family targets; d) p63; and e) E-cadherin.

In further aspects, there may be provided a system comprising: a data storage device configured to store an expression profile of a cancer patient's cancer; a server in data communication with the data storage device, suitably programmed to analyze the expression profile by a predictive analytic, therefore generating a prognosis of the cancer patient. In a further aspect, the system is further configured to report the prognosis. The system may also include a graphic user interface for user input and/or prognosis output.

There may also be provided a tangible computer program product comprising a computer readable medium having computer usable program code executable to perform one or more operations, wherein the operations comprise analyzing the expression profile of a patient's cancer by a predictive analytic, therefore generating a prognosis of the cancer patient.

Embodiments discussed in the context of methods and/or compositions of the invention may be employed with respect to any other method or composition described herein. Thus, an embodiment pertaining to one method or composition may be applied to other methods and compositions of the invention as well.

As used herein the terms “encode” or “encoding” with reference to a nucleic ac are used to make the invention readily understandable by the skilled artisan; however, these terms may be used interchangeably with “comprise” or “comprising” respectively.

As used herein the specification, “a” or “an” may mean one or more. As used herein in the claim(s), when used in conjunction with the word “comprising”, the words “a” or “an” may mean one or inure than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: An exemplary embodiment of intelligent clinical decision support system for Yes versus No progress diagnosis based on CART decision tree (RQ: relative quantification values determined by real-time RT-PCR or gene expression array. The notation MW, LM, WC, are the initials of the individuals (technician) who performed the assay.)

FIG. 2: An exemplary embodiment of intelligent clinical decision support system for Yes versus No progress diagnosis based tin CART decision tree.

FIG. 3: An exemplary embodiment of intelligent clinical decision support system for Yes versus No progress diagnosis based on CART decision tree.

FIG. 4: An exemplary embodiment of intelligent clinical decision support system for Yes versus No progress diagnosis based on CART decision tree.

FIG. 5: Progression Free Survival in two representative markers.

FIG. 6: Molecular Markers value assessment.

FIG. 7: Classification and Regression Tree Analysis for Molecular Marker determination of Clinical Progression in Bladder Cancer Patients.

FIG. 8: Classification and Regression Tree Analysis for Molecular Marker determination of Clinical Progression in Bladder Cancer Patients.

### DESCRIPTION OF ILLUSTRATIVE EMBODIMENTS

The instant invention overcomes several major problems with current cancer prognosis in providing methods, systems, and compositions using novel molecular biomarkers identified by expression profiling and clinical analysis of bladder cancer patients. Methods and systems of the present invention are optimal for patients determined to have cancer, in particular, epithelial cancer such as bladder cancer.

Certain aspects of the invention is based, in part, on the development of intelligent systems (based on artificial intelligence) called molecular i-Biomarkers that will predict clinical outcomes of patients with bladder cancer. The molecular markers (input for i-Biomarker system) are initially chosen based on biological knowledge and are based on signaling pathways. The signaling pathway is composed not only of various genes from the pathway but also includes other modulators, such as non-coding RNAs. The inventors performed data preprocessing and modeling using neural networks (NN), support vector machines (SVM), and decision trees and genetic programming (GP). Based on an original implementation of CART and GP the inventors detected non-linear relationships between the markers, which can be expressed as a set of rules or as mathematical equations that will predict 100% the output, which can be progression after standard therapy or combinations between targeted and standard therapies. The inventors have implemented these methods to a list of 13 markers that the inventors have identified, for example markers in the miR-200 pathway, and were able to predict with 100% bladder cancer progression, for patients that received standard therapy. This knowledge-based program may have a graphic interface and may be integrated into a clinical workflow.

Further embodiments and advantages of the invention are described below.

### I. DEFINITIONS

“Prognosis” refers to as a prediction of how a patient will progress, and whether there is a chance of recovery. “Cancer prognosis” generally refers to a forecast or prediction of the probable course or outcome of the cancer. As used herein, cancer prognosis includes the forecast or prediction of any one or more of the following: duration of survival of a patient susceptible to or diagnosed with a cancer, duration of recurrence-free survival, duration of progression-free survival of a patient susceptible to or diagnosed with a cancer, response rate in a group of patients susceptible to or diagnosed with a cancer, duration of response in a patient or a group of patients susceptible to or diagnosed with a cancer, and/or likelihood of metastasis and/or cancer progression in a patient susceptible to or diagnosed with a cancer. Prognosis also includes prediction of favorable responses to cancer treatments, such as a conventional cancer therapy.

By “subject” or “patient” is meant any single subject for which therapy is desired, including humans, cattle, dogs, guinea pigs, rabbits, chickens, and so on. Also intended to be included as a subject are any subjects involved in clinical research trials not showing any clinical sign of disease, or subjects involved in epidemiological studies, or subjects used as controls.

A good or had prognosis may, for example, be assessed in terms of patient survival, likelihood of disease recurrence, disease metastasis, or disease progression (patient survival, disease recurrence and metastasis may for example be assessed in relation to a defined time point, e.g. at a given number of years after cancer surgery (e.g. surgery to remove one or more tumors) or after initial diagnosis). In one embodiment, a good or had prognosis may be assessed in terms of overall survival, disease-free survival or progression-free survival.

In one embodiment, the marker level is compared to a reference level representing the same marker. In certain aspects, the reference level may be a reference level of expression from non-cancerous tissue from the same subject. Alternatively, reference level may be a reference level of expression from a different subject or group of subjects. For example, the reference level of expression may be an expression level obtained from tissue of a subject or group of subjects without cancer, or an expression level obtained from non-cancerous tissue of a subject or group of subjects with cancer. The reference level may be a single value or may be a range of values. The reference level of expression can be determined using any method known to those of ordinary skill in the art. In some embodiments, the reference level is an average level of expression determined from a cohort of subjects with cancer. The reference level may also be depicted graphically as an area on a graph.

The reference level may comprise data obtained at the same time (e.g., in the same hybridization experiment) as the patient's individual data, or may be a stored value or set of values e.g. stored on a computer, or on computer-readable media. If the latter is used, new patient data for the selected marker(s), obtained from initial or follow-up samples, can be compared to the stored data for the same marker(s) without the need for additional control experiments.

The term “antibody” herein is used in the broadest sense and specifically covers intact monoclonal antibodies, polyclonal antibodies, multispecific antibodies (e.g. bispecific antibodies) formed from at least two intact antibodies, and antibody fragments.

The term “primer,” as, used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

### II. BIOMARKERS

The inventors have identified practical cancer prognostic biomarkers and developed methods, systems, and kits to use these markers for cancer prognosis or classification. For example, several biomarker genes, including miR-200 family members (e.g., miR-200b & c, miR-205, miR-429 and miR-141), direct miR-200 family targets (e.g., ZEB1, ZEB2, ZNF532, ERRFI-1), p63, CDH-1 (encoding E-cadherin) and TGF-α, were identified with expression patterns associated with prognosis, such as prediction of survival.

miRNAs are small ˜22 nucleotide RNAs that regulate gene expression post-transcriptionally in a sequence-specific manner to influence cell differentiation, survival and response to environmental cues. Each miRNA may regulate the expression of many target genes. Although highly homologous, the miR-200 family members (e.g., miR-141 (NCBI accession no. NR_{—}029682; SEQ ID NO:4) miR-429 (NCBI accession no NR_{—}029957, SEQ ID NO:5), miR-200a (NCBI accession no NR_{—}029834; SEQ ID NO:1), miR-200b (NCBI accession no. NR_{—}029639; SEQ ID NO:2) and miR-2000 (NCBI accession no NR_{—}029779 SEQ ID NO:3)) can be divided into two functional groups based on their seed sequences, nucleotides 2 to 7 of the miRNA, which play an important role in target recognition. The 2 groups differ by a single seed nucleotide—miR-200b, miR-429 and miR-200c share the 5′-AAUACU-3′ seed sequence and miR-200a and miR-141 have the 5′-AACACU-3′ seed. In addition, they are encoded from 2 gene clusters in mice—miR-200c and miR-141 on chromosome 6 and miR-200b, miR-200a and miR-429 on chromosome 4.

Zinc finger E-box-binding homeobox 1 is a protein that in humans is encoded by the ZEB1 gene (see, e.g., NCBI accession no NM_{—}001128128, SEQ ID NO:9). ZEB1 (also known as AREB6; BZP; MGC133261; NIL-2-A; NIL-2A; TCF8; ZEB; ZFHEP; ZFHX1A) encodes a human zinc finger transcription factor that represses T-Iymphocyte-specific IL2 gene expression by binding to a negative regulatory domain 100 nucleotides 5-prime of the IL2 transcription start site. Mutations of the gene are linked to posterior polymorphous corneal dystrophy 3.

Zinc finger E-box-binding homeobox 2 is a protein that in humans is encoded by the ZEB2 gene (see, e.g., NCBI accession no NM_{—}014795, SEQ ID NO:10). The ZEB2 gene (also known as SIP1; SIP-1; KIAA0569; SMADIP1; ZFHX1B) is a member of the delta-EF1 (TCF8)/Zfh1 family of 2-handed zinc finger/homeodomain proteins. ZEB2 interacts with receptor-mediated, activated full-length SMAD proteins. Mutations in the ZEB2 gene is associated with the Mowat-Wilson syndrome.

This gene ZNF532 maps on chromosome 18, at 18q21.32 according to Entrez Gene. In AceView, it covers 123.88 kb, from 54680811 to 54804694 (NCBI 36, March 2006), on the direct strand (see, e.g., NCBI accession no. NM_{—}018181; SEQ ID NO:11). The gene is also known as ZNF532, FLJ10697 or LOC55205, swarzaby. It has been described as zinc finger protein 532. This gene's in vivo function is yet unknown.

ERBB receptor feedback inhibitor 1 is a protein that in humans is encoded by, the ERRFI-1 gene (see, e.g., NCBI accession no. NM_{—}018948; SEQ ID NO:12). ERRFI-1 (also known as MIG6; GENE-33; MIG-6; RALT) is a cytoplasmic protein whose expression is upregulated with cell growth. It shares significant homology with the protein product of rat gene-33, which is induced during cell stress and mediates cell signaling.

Although the most ancient member of the p53 family, p63 is the most recently discovered and the least is known about this family member (Westfall and Pietenpol, 2004; see, e.g., NCBI accession no. NM_{—}003722; SEQ ID NO:7). Unlike p53, whose protein expression is not readily detectable in epithelial cells unless they are exposed to various stress conditions, p63 is expressed in select epithelial cells at high levels under normal conditions. p63 is highly expressed in embryonic ectoderm and in the nuclei of basal regenerative cells of many epithelial tissues in the adult including skin, breast myoepithelium, oral epithelium, prostate and urothelia. In contrast to the tumor suppressive function of p53, over-expression of select p63 splice variants is observed in many squamous carcinomas suggesting that p63 may act as an oncogene.

Cadherins (Calcium dependent adhesion molecules) are a class of type-1 transmembrane proteins. They play important roles in cell adhesion, ensuring that cells within tissues are bound together. They are dependent on calcium (Ca2+) ions to function, hence their name. E-cadherin (epithelial) is the most well-studied member of the family. It consists of 5 cadherin repeats (EC1˜EC5) in the extracellular domain, one transmembrane domain, and an intracellular domain that binds p120-catenin and beta-catenin (see, e.g., NCBI accession no. NM_{—}004360; SEQ ID NO:8). The intracellular domain contains a highly-phosphorylated region vital to beta-catenin binding and therefore to E-cadherin function. In epithelial cells, E-cadherin-containing cell-to-cell junctions are often adjacent to actin-containing filaments of the cytoskeleton.

Transforming growth factor alpha (TGF-α; see, e.g., NCBI accession no. NM_{—}001099691; SEQ ID NO:6) is upregulated in some human cancers. It is produced in macrophages, brain cells, and keratinocytes, and induces epithelial development. It is closely related to EGF, and can also bind to the EGF receptor with similar effects. TGFα stimulates neural cell proliferation in the adult injured brain. TGFα was cited in the 2001 NIH Stem Cell report to the U.S. Congress as promising evidence for the ability of adult stem cells to restore function in neurodegenerative disorders.

### III. INTELLIGENT SYSTEMS FOR CANCER PROGNOSIS

Creation of an intelligent system based on artificial intelligence, capable to predict clinical outcome with accuracy reaching 100% and taking as input a panel of molecular factors chosen through biological knowledge. Classification and Regression Trees (CART; see, e.g., Brennan et al. 1984, incorporated herein by reference) decision trees (DT; see e.g., Koza 1992, incorporated herein by reference) and Genetic Programming (GP) are the methods the inventors used to analyze the data. An original implementation of a DT and a GP system resulted into a modellequation using only a few molecular markers that created a model with 100% predictive accuracy for bladder cancer progression. This methodology can be adapted to various clinical questions that relate to outcomes after standard therapy or predict the best therapeutic combination for the hest clinical outcome. Multiple systems which correspond to specific clinical questions may be implemented. Based on an original program, it can expand to include imaging data as a more objective quantification of relapse/progression criteria or as a measure of tissue modification (3D measurement and optical density variations).

To the best of the inventors' knowledge this is the first time when intelligent systems combining molecular markers based on coding and non-coding RNAs and describing specific pathways are used to predict bladder cancer progression with such high accuracy. The intelligent system that results is very easy to use and intuitive with a graphic interface. The results are given in a few seconds. The cost will include molecular markers included in the equation and per patient fee for using the system.

### IV. EXPRESSION ASSESSMENT

In certain aspects, this invention entails measuring expression of one or more prognostic biomarkers in a sample of cells from a subject with cancer. The expression information may be obtained by testing cancer samples by a lab, a technician, a device, or a clinician. In a certain embodiment, the differential expression of one or more biomarkers including a miR-200 family member, a miR-200 family target and an epithelial marker may be measured.

The pattern or signature of expression in each cancer sample may then be used to generate a cancer prognosis or classification, such as predicting cancer survival or recurrence. The level of expression of a biomarker may be increased or decreased in a subject relative to a reference level. The expression of a biomarker may be higher in long-term survivors than in short-term survivors. Alternatively, the expression of a biomarker may be higher in short-term survivors than in long-term survivors.

Expression of one or more of biomarkers identified by the inventors could be assessed to predict or report prognosis or prescribe treatment options for cancer patients, especially bladder cancer patients.

The expression of one or more biomarkers may be measured by a variety of techniques that are well known in the art. Quantifying the levels of the messenger RNA (mRNA) of a biomarker may be used to measure the expression of the biomarker. Alternatively, quantifying the levels of the protein product of a biomarker may be to measure the expression of the biomarker. Additional information regarding the methods discussed below may be found in Ausubel et al., (2003) Current Protocols in Molecular Biology, John Wiley &amp; Sons, New York, N.Y., or Sambrook et al. (1989) Molecular Cloning: A Laboratory Manual, Cold Spring Harbor Press, Cold Spring Harbor, N.Y. One skilled in the art will know which parameters may be manipulated to optimize detection of the mRNA or protein of interest.

A nucleic acid microarray may be used to quantify the differential expression of a plurality of biomarkers. Microarray analysis may be performed using commercially available equipment, following manufacturer's protocols, such as by using the Affymetrix GeneChip® technology (Santa Clara, Calif.) or the Microarray System from Incyte (Fremont, Calif.). Typically, single-stranded nucleic acids (e.g., cDNAs or oligonucleotides) are plated, or arrayed, on a microchip substrate. The arrayed sequences are then hybridized with specific nucleic acid probes from the cells of interest. Fluorescently labeled cDNA probes may be generated through incorporation of fluorescently labeled deoxynucleotides by reverse transcription of RNA extracted from the cells of interest. Alternatively, the RNA may be amplified by in vitro transcription and labeled with a marker, such as biotin. The labeled probes are then hybridized to the immobilized nucleic acids on the microchip under highly stringent conditions. After stringent washing to remove the non-specifically bound probes, the chip is scanned by confocal laser microscopy or by another detection method, such as a CCD camera. The raw fluorescence intensity data in the hybridization files are generally preprocessed with the robust multichip average (RMA) algorithm to generate expression values.

Quantitative real-time PCR (qRT-PCR) may also be used to measure the differential expression of a plurality of biomarkers. In qRT-PCR, the RNA template is generally reverse transcribed into cDNA, which is then amplified via a PCR reaction. The amount of PCR product is followed cycle-by-cycle in real time, which allows for determination of the initial concentrations of mRNA. To measure the amount of PCR product, the reaction may be performed in the presence of a fluorescent dye, such as SYBR Green, which binds to double-stranded DNA. The reaction may also be performed with a fluorescent reporter probe that is specific for the DNA being amplified.

A non-limiting example of a fluorescent reporter probe is a TaqMan® probe (Applied Biosystems, Foster City, Calif.). The fluorescent reporter probe fluoresces when the quencher is removed during the PCR extension cycle. Multiplex qRT-PCR may be performed by using multiple gene-specific reporter probes, each of which contains a different fluorophore. Fluorescence values are recorded during each cycle and represent the amount of product amplified to that point in the amplification reaction. To minimize errors and reduce any sample-to-sample variation, qRT-PCR is typically performed using a reference standard. The ideal reference standard is expressed at a constant level among different tissues, and is unaffected by the experimental treatment.

Suitable reference standards include, but are not limited to, mRNAs for the housekeeping genes glyceraldehyde-3-phosphate-dehydrogenase (GAPDH) and β-actin. The level of mRNA in the original sample or the fold change in expression of each biomarker may be determined using calculations well known in the art.

Immunohistochemical staining may also be used to measure the differential expression of a plurality of biomarkers. This method enables the localization of a protein in the cells of a tissue section by interaction of the protein with a specific antibody. For this, the tissue may be fixed in formaldehyde or another suitable fixative, embedded in wax or plastic, and cut into thin sections (from about 0.1 mm to several mm thick) using a microtome. Alternatively, the tissue may be frozen and cut into thin sections using a cryostat. The sections of tissue may be arrayed onto and affixed to a solid surface (i.e., a tissue microarray). The sections of tissue are incubated with a primary antibody against the antigen of interest, followed by washes to remove the unbound antibodies. The primary antibody may be coupled to a detection system, or the primary antibody may be detected with a secondary antibody that is coupled to a detection system. The detection system may be a fluorophore or it may be an enzyme, such as horseradish peroxidase or alkaline phosphatase, which can convert a substrate into a colorimetric, fluorescent, or chemiluminescent product. The stained tissue sections are generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for the biomarker.

An enzyme-linked immunosorbent assay, or ELISA, may be used to measure the differential expression of a plurality of biomarkers. There are many variations of an ELISA assay. All are based on the immobilization of an antigen or antibody on a solid surface, generally a microtiter plate. The original ELISA method comprises preparing a sample containing the biomarker proteins of interest, coating the wells of a microtiter plate with the sample, incubating each well with a primary antibody that recognizes a specific antigen, washing away the unbound antibody, and then detecting the antibody-antigen complexes. The antibody-antibody complexes may be detected directly. For this, the primary antibodies are conjugated to a detection system, such as an enzyme that produces a detectable product. The antibody-antibody complexes may be detected indirectly. For this, the primary antibody is detected by a secondary antibody that is conjugated to a detection system, as described above. The microtiter plate is then scanned and the raw intensity data may be converted into expression values using means known in the art.

An antibody microarray may also be used to measure the differential expression of a plurality of biomarkers. For this, a plurality of antibodies is arrayed and covalently attached to the surface of the microarray or biochip. A protein extract containing the biomarker proteins of interest is generally labeled with a fluorescent dye.

The labeled biomarker proteins may be incubated with the antibody microarray. After washes to remove the unbound proteins, the microarray is scanned. The raw fluorescent intensity data maybe converted into expression values using means known in the art.

Luminex multiplexing microspheres may also be used to measure the differential expression of a plurality of biomarkers. These microscopic polystyrene beads are internally color-coded with fluorescent dyes, such that each bead has a unique spectral signature (of which there are up to 100). Beads with the same signature are tagged with a specific oligonucleotide or specific antibody that will bind the target of interest (i.e., biomarker mRNA or protein, respectively). The target, in turn, is also tagged with a fluorescent reporter. Hence, there are two sources of color, one from the bead and the other from the reporter molecule on the target. The beads are then incubated with the sample containing the targets, of which up 100 may be detected in one well. The small size/surface area of the beads and the three dimensional exposure of the beads to the targets allows for nearly solution-phase kinetics during the binding reaction. The captured targets are detected by high-tech fluidics based upon flow cytometry in which lasers excite the internal dyes that identify each bead and also any reporter dye captured during the assay. The data from the acquisition files may be converted into expression values using means known in the art.

In situ hybridization may also be used to measure the differential expression of a plurality of biomarkers. This method permits the localization of mRNAs of interest in the cells of a tissue section. For this method, the tissue may be frozen, or fixed and embedded, and then cut into thin sections, which are arrayed and affixed on a solid surface. The tissue sections are incubated with a labeled antisense probe that will hybridize with an mRNA of interest. The hybridization and washing steps are generally performed under highly stringent conditions. The probe may be labeled with a fluorophore or a small tag (such as biotin or digoxigenin) that may be detected by another protein or antibody, such that the labeled hybrid may be detected and visualized under a microscope. Multiple mRNAs may be detected simultaneously, provided each antisense probe has a distinguishable label. The hybridized tissue array is generally scanned under a microscope. Because a sample of tissue from a subject with cancer may be heterogeneous, i.e., some cells may be normal and other cells may be cancerous, the percentage of positively stained cells in the tissue may be determined. This measurement, along with a quantification of the intensity of staining, may be used to generate an expression value for each biomarker.

### V. CANCER TREATMENTS

In certain aspects, there may be provided methods for treating a subject determined to have cancer and with a predetermined expression profile of one or more biomarkers disclosed herein.

In a further aspect, biomarkers and related systems that can establish a prognosis of cancer patients in this invention can be used to identify patients who may get benefit of conventional single or combined modality therapy. In the same way, the invention can identify those patients who do not get much benefit from such conventional single or combined modality therapy and can offer them alternative treatment(s).

In certain aspects of the present invention, conventional cancer therapy may be applied to a subject wherein the subject is identified or reported as having a good prognosis based on the assessment of the biomarkers as disclosed. On the other hand, at least an alternative cancer therapy may be prescribed, as used alone or in combination with conventional cancer therapy, if a poor prognosis is determined by the disclosed methods, systems, or kits.

Conventional cancer therapies include one or more selected from the group of chemical or radiation based treatments and surgery. Chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

Radiation therapy that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part, of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Laser therapy is the use of high-intensity light to destroy tumor cells. Laser therapy affects the cells only in the treated area. Laser therapy may be used to destroy cancerous tissue and relieve a blockage in the esophagus when the cancer cannot be removed by surgery. The relief of a blockage can help to reduce symptoms, especially swallowing problems.

Photodynamic therapy (PDT), a type of laser therapy, involves the use of drugs that are absorbed by cancer cells; when exposed to a special light the drugs become active and destroy the cancer cells. PDT may be used to relieve symptoms of esophageal cancer such as difficulty swallowing.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

Alternative cancer therapy include any cancer therapy other than surgery, chemotherapy and radiation therapy in the present invention, such as immunotherapy, gene therapy, hormonal therapy or a combination thereof. Subjects identified with poor prognosis using the present methods may not have favorable response to conventional treatment(s) alone and may be prescribed or administered one or more alternative cancer therapy per se or in combination with one or more conventional treatments.

For example, the alternative cancer therapy may be a targeted therapy. The targeted therapy may be an anti-EGFR treatment. In one embodiment of the method of the invention, the anti-EGFR agent used is a tyrosine kinase inhibitor. Examples of suitable tyrosine kinase inhibitors are the quinazoline derivatives described in WO 96/33980, in particular gefitinib (Iressa). Other examples include quinazoline derivatives described in WO 96/30347, in particular erlotinib (Tarceva), dual EGFR/HER2 tyrosine kinase inhibitors, such as lapatinib, or pan-Erb inhibitors. In a preferred embodiment of the method or use of the invention, the anti-EGFR agent is an antibody capable of binding to EGFR, i.e. an anti-EGFR antibody.

In a further embodiment, the anti-EGFR antibody is an intact antibody, i.e. a full-length antibody rather than a fragment. An anti-EGFR antibody used in the method of the present invention may have any suitable affinity and/or avidity for one or more epitopes contained at least partially in EGFR. Preferably, the antibody used binds to human EGFR with an equilibrium dissociation constant (K0) of 10″8 M or less, more preferably 10˜10 M or less.

Particularly antibodies for use in the present invention include zalutumumab (2F8), cetuximab (Erbitux), nimotuzumab (h-R3), panitumumab (ABX EGF), and matuzumab (EMD72000), or a variant antibody of any of these, or an antibody which is able to compete with any of these, such as an antibody recognizing the same epitope as any of these. Competition may be determined by any suitable technique. In one embodiment, competition is determined by an ELISA assay. Often competition is marked by a significantly greater relative inhibition than 5% as determined by ELISA analysis.

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Gene therapy is the insertion of polynucleotides, including DNA or RNA, into an individual's cells and tissues to treat a disease. Antisense therapy is also a form of gene therapy in the present invention. A therapeutic polynucleotide may be administered before, after, or at the same time of a first cancer therapy. Delivery of a vector encoding a variety of proteins is encompassed within the invention. For example, cellular expression of the exogenous tumor suppressor oncogenes would exert their function to inhibit excessive cellular proliferation, such as p53, p16 and C-CAM.

Additional agents to be used to improve the therapeutic efficacy of treatment include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

### VI. KITS

Certain aspects of the present invention also encompass kits for performing the diagnostic and prognostic methods of the invention. Such kits can be prepared from readily available materials and reagents. For example, such kits can comprise any one or more of the following materials: enzymes, reaction tubes, buffers, detergent, primers, probes, antibodies. In a preferred embodiment, these kits allow a practitioner to obtain samples of neoplastic cells in blood, tears, semen, saliva, urine, tissue, serum, stool, sputum, cerebrospinal fluid and supernatant from cell lysate. In another preferred embodiment these kits include the needed apparatus for performing RNA extraction, RT-PCR, and gel electrophoresis. Instructions for performing the assays can also be included in the kits.

In a particular aspect, these kits may comprise a plurality of agents for assessing the differential expression of a plurality of biomarkers, for example, one or more miR-200 family members or targets in combination with TGFalpha, wherein the kit is housed in a container. The kits may further comprise instructions for using the kit for assessing expression, means for converting the expression data into expression values and/or means for analyzing the expression values to generate prognosis. The agents in the kit for measuring biomarker expression may comprise a plurality of PCR probes and/or primers for qRT-PCR and/or a plurality of antibody or fragments thereof for assessing expression of the biomarkers. In another embodiment, the agents in the kit for measuring biomarker expression may comprise an array of polynucleotides complementary to the mRNAs of the biomarkers of the invention. Possible means for converting the expression data into expression values and for analyzing the expression values to generate scores that predict survival or prognosis may be also included.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit of the invention will typically comprise the container described above and one or more other containers comprising materials desirable from a commercial and user standpoint, including buffers, diluents, filters, needles, syringes, and package inserts with instructions for use.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Bladder Cancer Prognosis

Patients and Methods

One hundred thirty seven patients were included in this study. These patients were diagnosed with bladder cancer and undergone surgeries like TUR (96/137=70.0730%), nephrectomy (7/137=5.1095%) or cystectomy (34/137=24.8175%). There were 8 patients (5.8394%) who presented clinical detectable metastasis. The mean age of the patients was 67.35 years, the minimum age being 36.5 years and the maximum age 92.6 years.

Through the following presented experiments the inventors wanted to be able to predict the progress of the disease with the help of the studied factors.

Patients Data

In the inventors' dataset there are 2 different types of factors: clinicopathological factors and the genes. There are thirteen genes which influence the evolution of the disease, and below there is a table which presents some basic statistics about them.

TABLE 1
Skewness and kurtosis ratio testing the normality of the variables

| | | Skew- | | | | |
|---|---|---|---|---|---|---|
| | Skew- | ness | | Kurtosis | Skewness | Kurtosis |
| Variable | ness | S.E. | Kurtosis | S.E. | Ratio | Ratio |
| ZNF532 | −0.30 | 0.36 | −0.71 | 0.70 | −0.83 | −1.01 |
| array data | | | | | | |
| RQ mir 200c | 2.40 | 0.30 | 6.28 | 0.60 | 7.77 | 10.33 |
| RQ Zeb2 | 3.60 | 0.41 | 15.13 | 0.80 | 8.69 | 18.71 |
| RQ TGF- | 2.93 | 0.42 | 10.96 | 0.83 | 6.86 | 13.16 |
| alpha | | | | | | |
| RQ ERRFI-1 | 1.38 | 0.42 | 2.31 | 0.83 | 3.23 | 2.78 |
| RQ ZNF-532 | 0.46 | 0.42 | −0.54 | 0.83 | 1.09 | −0.65 |
| RQ mir141 | 2.36 | 0.41 | 5.5 | 0.80 | 5.71 | 6.89 |
| RQ mir 429 | 2.94 | 0.41 | 8.96 | 0.80 | 7.12 | 11.07 |
| RQ mir 205 | 1.44 | 0.41 | 1.11 | 0.80 | 3.49 | 1.37 |
| RQ mir 200b | 2.99 | 0.41 | 10.35 | 0.80 | 7.23 | 12.79 |
| RQ CDH1 | 0.97 | 0.41 | 0.59 | 0.80 | 2.35 | 0.73 |
| RQ Zeb1 | 1.86 | 0.41 | 3.06 | 0.80 | 4.5 | 3.79 |
| RQ p63 | 0.99 | 0.41 | 0.06 | 0.80 | 2.41 | 0.082 |

Abbreviations:
S.E.: standard error

TABLE 2
Pearson correlation coefficients

| | ZNF532 | RQ | RQ | RQ | RQ | RQ | | RQ | RQ | RQ | | | |
|---|---|---|---|---|---|---|---|---|---|---|---|---|---|
| | array | mir | Zeb2 | TGF- | ERRFI- | ZNF- | RQ | mir | mir | mir | RQ | RQ | RQ |
| Variable | data | 200c | LM | alpha | 1 | 532 | mir141 | 429 | 205 | 200b | CDH1 | Zeb1 | p63 |
| ZNF532 | 1.00 | 0.05 | 0.46 | −0.20 | −0.39 | 0.44 | −0.09 | −0.06 | 0.35 | −0.16 | −0.31 | 0.39 | 0.32 |
| array data | | | | | | | | | | | | | |
| RQ mir | 0.05 | 1.00 | 0.17 | −0.11 | 0.03 | 0.10 | 0.86 | 0.90 | 0.53 | 0.78 | 0.15 | 0.22 | −0.15 |
| 200c | | | | | | | | | | | | | |
| RQ Zeb2 | 0.46 | 0.17 | 1.00 | 0.07 | 0.04 | 0.72 | 0.04 | 0.07 | −0.02 | −0.07 | −0.41 | 0.95 | −0.32 |
| RQ TGF- | −0.20 | −0.11 | 0.07 | 1.00 | 0.60 | 0.38 | −0.04 | −0.05 | −0.10 | 0.13 | −0.06 | 0.19 | −0.25 |
| alpha | | | | | | | | | | | | | |
| RQ | −0.39 | 0.03 | 0.04 | 0.60 | 1.00 | 0.07 | 0.12 | 0.04 | −0.21 | 0.22 | 0.08 | 0.07 | −0.35 |
| ERRFI-1 | | | | | | | | | | | | | |
| RQ ZNF- | 0.44 | 0.10 | 0.72 | 0.38 | 0.07 | 1.00 | 0.01 | 0.07 | 0.04 | −0.03 | −0.40 | 0.76 | −0.17 |
| 532 | | | | | | | | | | | | | |
| RQ mir141 | −0.09 | 0.86 | 0.04 | −0.04 | 0.12 | 0.01 | 1.00 | 0.90 | 0.43 | 0.76 | 0.16 | 0.10 | −0.05 |
| RQ mir | −0.06 | 0.90 | 0.07 | −0.05 | 0.04 | 0.07 | 0.90 | 1.00 | 0.32 | 0.90 | 0.19 | 0.13 | −0.19 |
| 429 | | | | | | | | | | | | | |
| RQ mir | 0.35 | 0.53 | −0.02 | −0.10 | −0.21 | 0.04 | 0.43 | 0.32 | 1.00 | 0.25 | −0.09 | −0.00 | 0.48 |
| 205 | | | | | | | | | | | | | |
| RQ mir | −0.16 | 0.78 | −0.07 | 0.13 | 0.22 | −0.03 | 0.76 | 0.90 | 0.25 | 1.00 | 0.18 | −0.03 | −0.21 |
| 200b | | | | | | | | | | | | | |
| RQ CDH1 | −0.31 | 0.15 | −0.41 | −0.06 | 0.08 | −0.40 | 0.16 | 0.19 | −0.09 | 0.18 | 1.00 | −0.33 | 0.12 |
| RQ Zeb1 | 0.39 | 0.22 | 0.95 | 0.19 | 0.07 | 0.76 | 0.10 | 0.13 | −0.00 | −0.03 | −0.33 | 1.00 | −0.29 |
| RQ p63 | 0.32 | −0.15 | −0.32 | −0.25 | −0.35 | −0.17 | −0.05 | −0.19 | 0.48 | −0.21 | 0.12 | −0.29 | 1.00 |

Marked correlations are significant at p < 0.5

Preprocessing

Skewness ratio, i.e., skewness ratio=skewness/(skewness standard error) and kurtosis ratio, i.e., kurtosis ratio=kurtosis/(kurtosis standard error) were used to estimate if the data is normally distributed. Skewness or kurtosis ratio less than −2 or greater than 2 indicate deviation from normality. Parametric statistical methods, that use means and standard deviations, such as Student's t-test or Shapiro-Wilk test, if applied to data that is not normally distributed, will provide weaker results then non-parametric methods, e.g., classification trees (see for example, Nisbet et al., 2009, incorporated herein by reference).

Result

In order to predict the progress of the patients' disease the inventors used one of the most known AI methods, which is CART. The data was randomly split into a training set (50% patients) and a testing set (50% patients) and the reported error is on test set.

The inventors used the following main settings for the CART algorithm: the goodness-of-fit measure was the GINI index, the prior class probabilities were estimated from data, the stopping option for pruning was misclassification error and the minimum number of patients per node, controlling when split selection stops and pruning begins, was five.

The results that were obtained using CART have 100% accuracy and so, there is no reason to present the specificity and sensitivity or ROC curve etc.

For more accurate results after the input of new data, one of the methods the inventors tried was combining more CART trees with 100% accuracy into a vote of confidence model, the ensemble giving the most voted response.

For the first experiment the inventors used as inputs the following genes, after the inventors removed some genes that were highly correlated, using the Pearson correlations. The output was the variable Progression Yes/No).

TABLE 3
Descriptive Statistics of the Data

| | Descriptive Statistics (date_anderson) | | | | |
|---|---|---|---|---|---|
| Variable | Valid N | Mean | Minimum | Maximun | Std. Dev. |
| ZNF532 array data | 43 | 7.7241 | 6.441284 | 8.893 | 0.643 |
| RQ mir 200c | 60 | 258.7756 | 1.000000 | 1417.955 | 305.254 |
| RQ Zeb2 | 32 | 11.9019 | 1.000000 | 96.020 | 18.173 |
| RQ TGF~alpha | 30 | 17.0514 | 1.000000 | 104.152 | 20.473 |
| RQ ERRFI-1 | 30 | 7.9950 | 1.000000 | 27.529 | 5.998 |
| RQ ZNF-532 | 30 | 3.7109 | 1.000000 | 8.430 | 2.082 |
| RQ mir 205 | 32 | 924.8561 | 1.000000 | 3780.226 | 1071.609 |
| RQ mir 200b | 32 | 357.1446 | 1.000000 | 2781.438 | 572.803 |
| RQ CDH1 | 32 | 93.9271 | 1.000000 | 284.659 | 70.989 |
| RQ p63 | 32 | 141.9586 | 1.000000 | 431.649 | 125.003 |

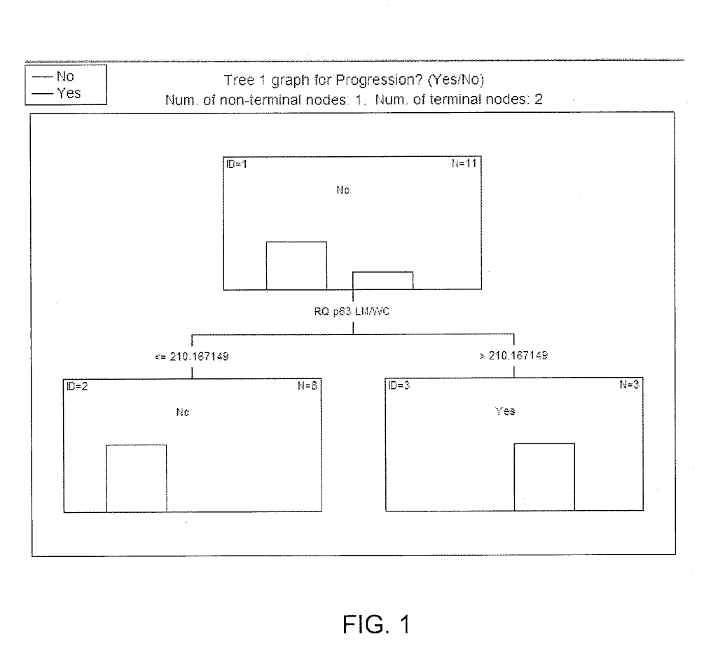

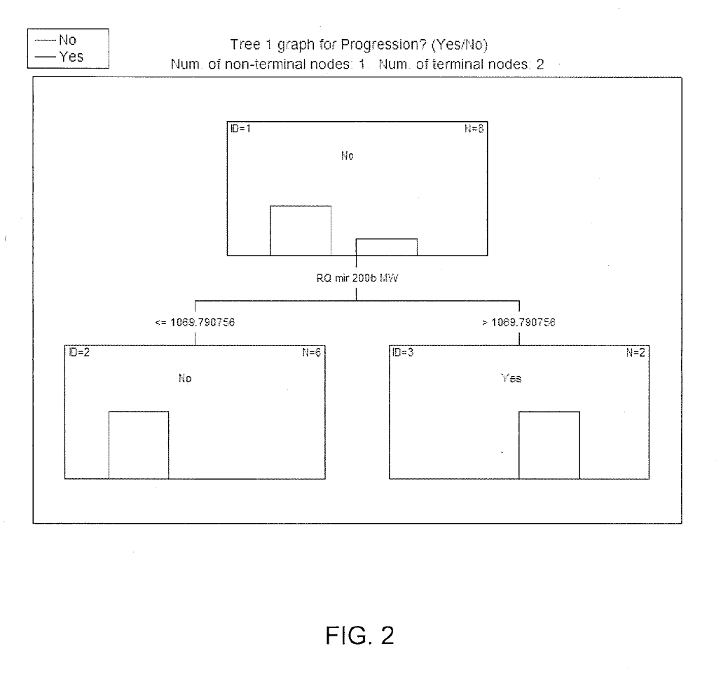

Processing the patient trough a classification tree is a very easy process. The patient “goes in” at the top of the tree (root) and the value of the first predictor (e.g., RQ mir 200b, FIG. 2) is compared with the cutoff value RQ mir 200b 1069.790756). Based on the corresponding tree rule (e.g., RQ mir 200b≦1069.790756 or RQ mir 200b>1069.790756), he advances trough the corresponding non-terminal nodes (blue node) towards a terminal node (red nodes) that will give his diagnosis. The two decision trees shown in FIGS. 1-2 selected the relevant predictors and discovered the relevant cutoff values from data. They can be read as easy to use “If/Then” rules, each corresponding to a particular tree branch.

The set of rules for the two diagnosis categories decision tree is the following (see FIG. 1):

If RQ p63≦210.167149 then the diagnosis is No;

If RQ p63>210.167149 then the diagnosis is Yes.

The rules of the first decision tree (see FIG. 2) are:

If RQ mir 200b≦1069.790756 then the diagnosis is No

If RQ mir 200b>1069.790756 then the diagnosis is No

For the next experiment with CART the inventors used the Chi-square feature selection method (see, Liu and Setiono 1995, incorporated herein by reference) to obtain the rank for each gene and the inventors selected the first five as inputs. The output remained the same. The 5 genes that were chosen are the following (Table 4):

TABLE 4
Five genes selected by feature selection

| | Best predictors for categorical dependent | |
|---|---|---|
| | var: Progression? (Yes/No) (date_anderson) | |
| | Chi-square | p-value |
| RQ TGF-alpha MW | 10.09286 | 0.072647 |
| RQ mir 200c MW | 6.28765 | 0.279227 |
| RQ mir 205 MW | 5.56092 | 0.351313 |
| RQ Zeb2 LM | 4.98042 | 0.289313 |
| RQ p63 LM/WC | 4.63915 | 0.461484 |

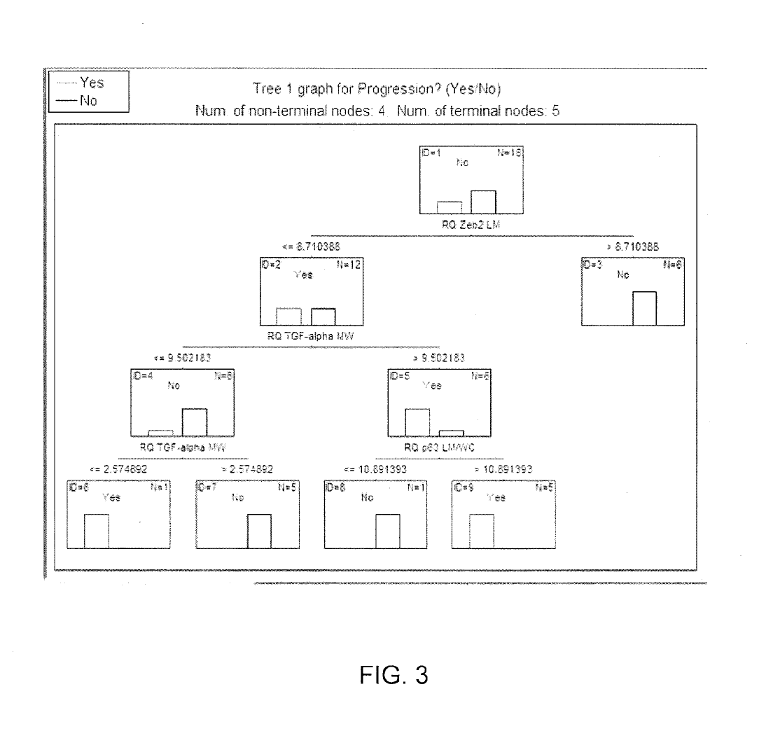

The rules of the decision tree presented in FIG. 3 are:

If RQ Zeb2≦8.710388 and RQ TGF-alpha MW≦9.502183 and RQ TGF-alpha MW≦2.574892 then the diagnosis is Yes

If RQ Zeb2≦8.710388 and RQ TGF-alpha MW≦9.502183 and RQ TGF-alpha MW>2.574892 then the diagnosis is No

If RQ Zeb2≦8.710388 and RQ TGF-alpha>9.502183 and RQ p63≦10.891393 then the diagnosis is No

If RQ Zeb2≦8.710388 and RQ TGF-alpha>9.502183 and RQ p63>10.891393 then the diagnosis is Yes

If RQ Zeb2>8.710388 then the diagnosis is No

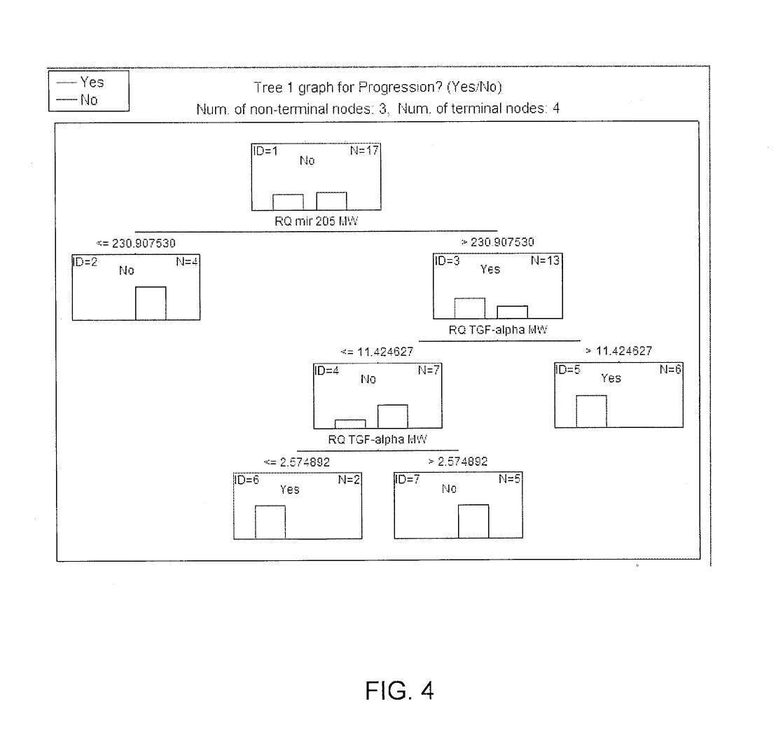

The rules of the decision tree presented in FIG. 4 are:

If RQ mir 205≦230.907530 then the diagnosis is No

If RQ mir 205>230.907530 and RQ TGF-alpha≦11.424647 and RQ TGF-alpha MW>2.574892 then the diagnosis is No

If RQ mir 205>230.907530 and RQ TGF-alpha≦11.424647 and RQ TGF-alpha≦2.574892 then the diagnosis is Yes

If RQ mir 205>230.907530 and RQ TGF-alpha>11.424647 then the diagnosis is Yes.
References: American Cancer Society. Cancer: Facts and Figures 2009; 19-20; Nisbet et al., 2009; Lai and Setiono 1995.

### Example 2

### Prognostic Significance of miR-200 Family in Bladder Cancer Progression

The MicroRNAs (miRs) are 20 to 25 nucleotide non-coding RNAs involved in many if not all biological functions, including cancer progression (Xi Y et al., 2006). The miR-200 family members became notorious for their demonstrated role in modulating the epithelial to mesenchymal transition (EMT) phenotype with important implications for cell migration/invasion (Gregory P A et al., 2008; Korpal M et al., 2008; Hurteau GJ et al., 2007). Recently the inventors reported that miR-200 family members are modulators of EGFR response and EMT in bladder cancer (Adam L et al., 2009). Further, the miR-200 family combined with a demonstrated tumor-promoting role of the EGFR-TGF-α axis in bladder cancer were all suggestive of a potential role in predicting clinical outcome in this type of cancer. To test this hypothesis, the inventors performed a retrospective study on 60 patients that had never received treatment prior to tumor tissue collection and investigated several EMT-related molecules by qRT-PCR.

The inventors have analyzed all five miR-200 family members (miR-200b and c, miR-205, miR-429 and miR-141), direct miR-200 family targets (ZEB1, ZEB2, ZNF532, ERRFI-1), p63, E-cadherin and TGF-α. Assessment is made in 32 patient tissues that had not received prior systemic therapy. All tissue analyzed was obtained from TUR specimens (Table 5).

TABLE 5
RT-PCR assessment of miR 200 Family, its targets and EMT Markers.

| miR 200 | | | Direct miR 200 | | | EMT/EGFR | | |
|---|---|---|---|---|---|---|---|---|
| Family | n | Mean ± SE | Family Targets | n | Mean ± SE | Markers | n | Mean ± SE |
| miR 200b | 32 | 357.1 ± 101.3 | Zeb1 | 32 | 8.7 ± 1.6 | p 63 | 32 | 142.0 ± 22.1 |
| miR 200c | 32 | 275.6 ± 68.9 | Zeb2 | 32 | 11.9 ± 3.2 | CDH-1 | 32 | 93.9 ± 12.5 |
| miR 205 | 32 | 924.9 ± 189.4 | ZNF-532 a-d | 30 | 3.7 ± 0.4 | TGF-a | 30 | 17.1 ± 3.7 |
| miR 429 | 32 | 302.1 ± 96.9 | ZNF-532 a & b | 32 | 7.7 ± 0.1 | | | |
| miR 141 | 32 | 407.2 ± 104.9 | ERRFI-1 | 30 | 8.0 ± 1.1 | | | |

Table 6 displays patient characteristics at tissue collection and status at latest follow-up. Median follow-up time was 8.5 months for the entire cohort. Progression was defined as advancing stage, or development of nodal or visceral metastases or recurrence of same stage (1 of 11 patients defined as progressed). NED=no evidence of disease, AWD=alive with disease, DOD=dead of disease.

TABLE 6
Patient Characteristics at Tissue Collection and Status at
Latest Follow-up

| Patient Characteristics at | | | | Disease Status at | | | | |
|---|---|---|---|---|---|---|---|---|
| Time of Tissue Collection | | | | Last Follow-up | | | | |
| | T | N | M | Initial | Prog- | | | |
| n | Stage | Stage | Stage | Stage | ressed | NED | AWD | DOD |
| 7 | T1 | 1 | 0 | T1 (n = 7) | 2 | 3 | 1 | 3 |
| 23 | T2 | 6 | 3 | T2 (n = 23) | 9 | 6 | 11 | 5 |
| 2 | T3-4 | 0 | 0 | T3-4 (n = 2) | 0 | 2 | 0 | 0 |

In general, the miR200 family directly correlated with each other and their targets (i.e. Zeb1, ZNF532) did the same. Red color demonstrates significance at p<0.05.

Table 7 displays correlation of all proposed molecular markers. In general, the miR200 family directly correlated with each other and their targets (i.e. Zeb1, ZNF532) did the same. Red color demonstrates significance at p<0.05.

TABLE 7
Correlation of all proposed molecular markers.

| | miR 200 Family | | | | | Direct miR 200 Family Targets | | | | | EMT/EGFR Markers | | |
|---|---|---|---|---|---|---|---|---|---|---|---|---|---|
| | miR | miR | miR | miR | miR | | | ZNF-532 | ZNF-532 | | p | | |
| | 200b | 200c | 205 | 429 | 141 | Zeb1 | Zeb2 | a-d | a & b | ERRFI-1 | 63 | CDH-1 | TGF-a |
| miR 200b | | 0.78 | 0.25 | 0.9 | 0.76 | −0.03 | −0.07 | −0.03 | −0.16 | 0.22 | −0.21 | 0.18 | 0.13 |
| miR 200c | 0.78 | | 0.53 | 0.9 | 0.86 | 0.22 | 0.17 | 0.1 | 0.05 | 0.03 | −0.15 | 0.15 | −0.11 |
| miR 205 | 0.25 | 0.53 | | 0.32 | 0.43 | 0 | −0.02 | 0.04 | 0.35 | −0.21 | 0.48 | −0.09 | −0.1 |
| miR 429 | 0.9 | 0.9 | 0.32 | | 0.9 | 0.13 | 0.07 | 0.07 | −0.06 | 0.04 | −0.19 | 0.19 | −0.05 |
| miR 141 | 0.76 | 0.86 | 0.43 | 0.9 | | 0.1 | 0.04 | 0.01 | −0.09 | 0.12 | −0.05 | 0.16 | −0.04 |
| Zeb1 | −0.03 | 0.22 | 0 | 0.13 | 0.1 | | 0.95 | 0.76 | 0.39 | 0.07 | −0.29 | −0.33 | 0.19 |
| Zeb2 | −0.07 | 0.17 | −0.02 | 0.07 | 0.04 | 0.95 | | 0.72 | 0.46 | 0.04 | −0.32 | −0.41 | 0.07 |
| ZNF-532 a-d | −0.03 | 0.1 | 0.04 | 0.07 | 0.01 | 0.76 | 0.72 | | 0.44 | 0.07 | −0.17 | −0.4 | 0.38 |
| ZNF-532 a & b | −0.16 | 0.05 | 0.35 | −0.06 | −0.09 | 0.39 | 0.46 | 0.44 | | −0.39 | 0.32 | −0.31 | −0.2 |
| ERRFI-1 | 0.22 | 0.03 | −0.21 | 0.04 | 0.12 | 0.07 | 0.04 | 0.07 | −0.39 | | −0.35 | 0.08 | 0.6 |
| p 63 | −0.21 | −0.15 | 0.48 | −0.19 | −0.05 | −0.29 | −0.32 | −0.17 | 0.32 | −0.35 | | 0.12 | −0.25 |
| CDH-1 | 0.18 | 0.15 | −0.09 | 0.19 | 0.16 | −0.33 | −0.41 | −0.4 | −0.31 | 0.08 | 0.12 | | −0.06 |
| TGF-alpha | 0.13 | −0.11 | −0.1 | −0.05 | −0.04 | 0.19 | 0.07 | 0.38 | −0.2 | 0.6 | −0.25 | −0.06 | |

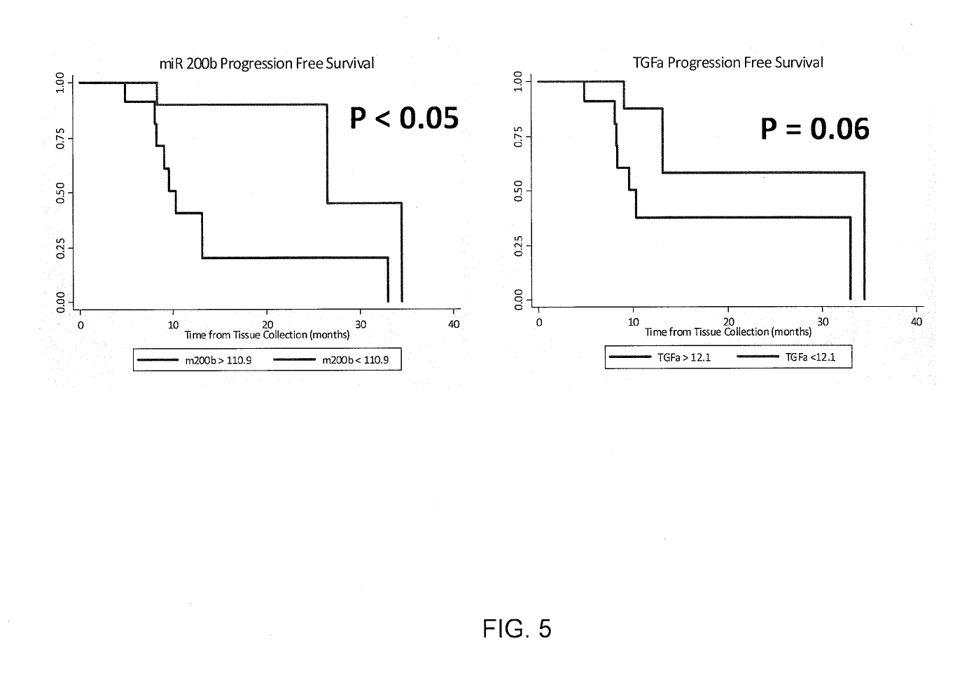

FIG. 5 shows progression free survival in two representative markers. Thirty-two patients make up the cohort with time listed, in months from TUR. HR for miR200b is 0.19 (0.04-0.95) and HR for TGF-α is 0.21 (0.04-1.08). This suggests that patient within this cohort who had elevated miR200 Family markers & TGF-α might be at greater risk for clinical progression.

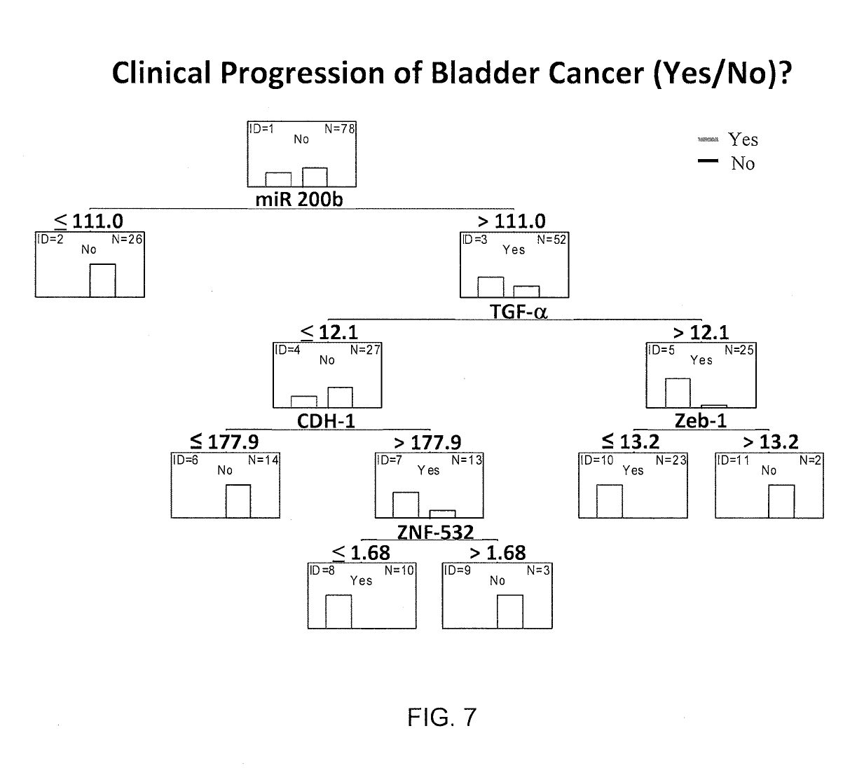

To determine the role of these biological markers as predictors of clinical outcome, the inventors tested the accuracy of predicting disease progression modeling by using various types of artificial intelligence agents: neural networks, support vector machines, and decision trees. The Classification and Regression Trees (CART) (e.g., as shown in FIG. 7) was the most accurate algorithm in all tests tested. It selected for the relevant predictors of progression and discovered the relevant cutoff values from the dataset on an “if then” rule set.

The inventors used the following CART algorithm settings: GINI index was used to measure the goodness of fit, the prior class probabilities were estimated from data, the stopping option for pruning was misclassification error, the minimum number of patients per node, controlling when split selection stops and pruning begins, was five. Thus, the CART decision tree selected the relevant predictors and discovered the relevant cutoff values from the dataset on an “if/then” rules set. The data was first resampled, to increase the number of patients, and then randomly split into training set (50%) and testing set (50%).

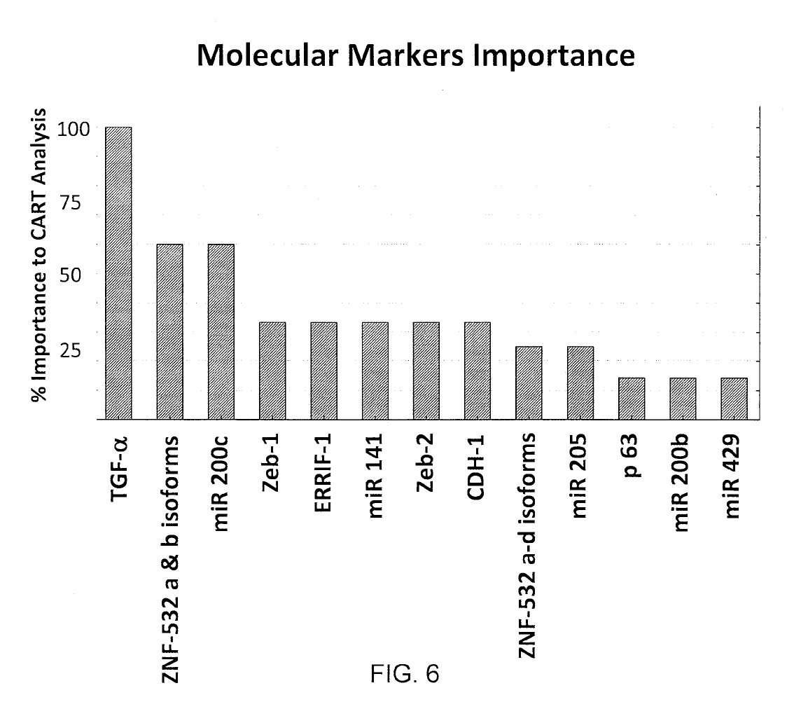

The inventors found that the most important predictors were: TGF-α, followed by ZEB1, miR-200c, ZEB2, ZNF532, p63 and ERRFI-1. FIG. 6 shows molecular markers value assessment. When utilizing AI analysis, several CART models were developed with accuracy between 90% and 100% (data not shown). Based on these models, a voting process was performed using the Ensemble method and an importance value estimate for each molecular marker is presented with regard to clinical progression. In the above figure, TGF-α was identified as the most important molecular marker.

Finally, the inventors obtained a five-non-terminal- and six terminal-nodes decision tree which could predict the bladder cancer progression with 100% accuracy in this dataset. Most importantly, this type of analysis allows for a continuous inclusion of new data until an “input saturation” is achieved in which the decision tree and the cutoffs of each of the predictors will remain unchanged. FIG. 7 shows classification and regression tree analysis for molecular marker determination of clinical progression in bladder cancer patients. This figure represents one of the proposed models that contributed to development of FIG. 6. This model had a predicted accuracy of 100% for this patient cohort. Interestingly, this model suggests that elevated miR200 expression combined with ↑TGF-α & ↓Zeb1 may define a subgroup of patients with worse clinical outcome.

In biological terms, the inventors found that patients with bladder tumors reminiscent of an “epithelial phenotype” (higher miR-200, lower ZEB1, higher E-cadherin and p63) that also express high levels of TGF-α are most likely to progress over time.

Importantly, this particular “epithelial” phenotype could also be found in the inventors' in vitro cellular models of bladder cancer, a typical example being the 253J-P and 253J-BV. The 253J-P cells, are non-tumorigenic when implanted orthotopically in mice whereas 253J BV represent its tumorigenic derivative after five cycles of orthotopic mouse implantation. 253J BV cells are characterized by a 70% tumorigenicity, express higher mill-200b, developed an autocrine loop for TGF-α and express higher levels E-cadherin, despite the fact that both cell lines co-express vimentin. Altogether, these results suggest that miR-200 and TGF-α signaling are important phenotypic modulators of bladder cancer progression, which hold promising clinical outcome predictor values.

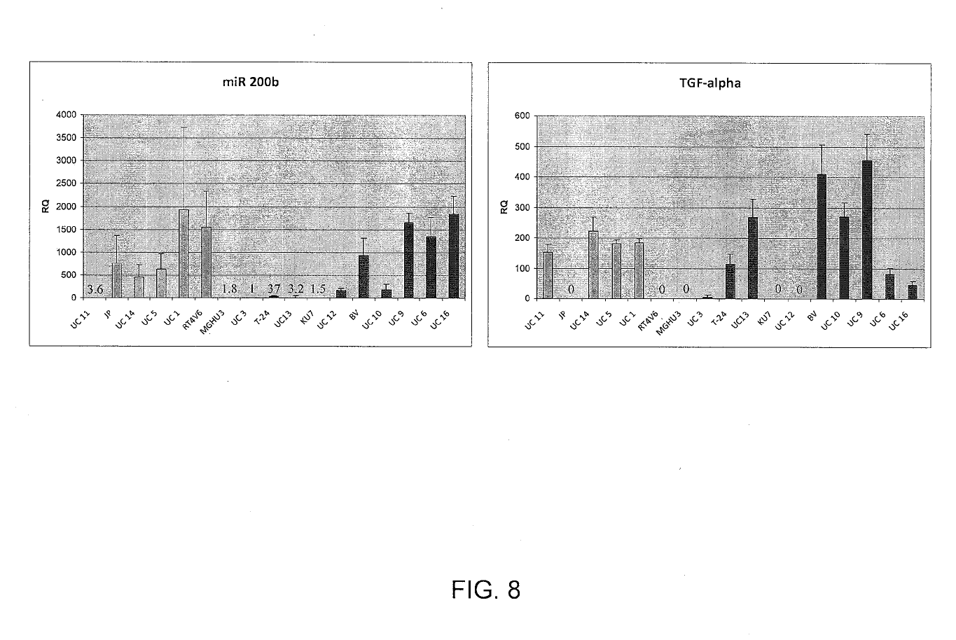

FIG. 8 shows miR 200b & TGF-α expression based on invasion status in UC cell lines. In general, TGF-α is expressed more in epithelial lines (as defined by CDH-1 expression) as compared to mesenchymal. Further, the most invasive epithelial lines express higher levels of TGF-α as compared to their non-invasive counterparts (e.g. BV & UC9 v, JP & RT4V6). Blue color represents non-invasive cell lines while red color represents invasive status. Invasion status based on 48 h results through matrigel >20%.

The inventors' results suggest that the miR-200 family and TGF-α signaling are important phenotypic modulators of bladder cancer progression and hold promise as new molecular markers for predicting clinical outcomes.

Materials

Approval for this study was obtained via the Institutional Review Board at MD Anderson Cancer Center.

RNA was extracted from frozen patient tumors and urothelial cell lines. It was then normalized to a concentration of 2 ng/μL.

In-vitro invasion assays with matrigel were performed on all urothelial cell lines. The inventors defined invasion as >20% invasion at 48-hours.

RT-PCR was performed utilizing TaqMan® Reagents (Applied Biosystems) for the following molecular markers: miR-200 family members (miR-200b & c, miR-205, miR-429 and miR-141), direct miR-200 family targets (ZEB1, ZEB2, ZNF532, ERRFI-1), p63, E-cadherin and TGF-α.

Traditional statistical analyses were performed to determine progression free survival utilizing Cox Proportional Hazard Models with P<0.05 being significant.

After traditional statistics identified possible interactions, the inventors then identified data for inclusion in predictive models. To that end, the inventors aimed to assess the role of these biological markers as predictors of clinical outcome, and tested the accuracy of predicting disease progression models by using various types of artificial intelligence agents: neural networks, support vector machines, genetic programming, and decision trees.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1) Xi Y et al. Biomarker Insights 2006; 1:113-21
2) Gregory P A et al. Nature Cell Biology 2008, 10(5):593-601
3) Korpal M etal. Journal of Biological Chemistry 2008; 283(22):14910-14
4) Hurteau G J et al. Cancer Research 2007; 67(17):7972-76
5) Adam L et al. Clinical Cancer Research 2009; 15(16):5060-72
6) Westfall and Pietenpol, Carcinogenesis, Vol, 25, No 6, 857-864, 2004
7) Breiman et al. Classification and Regression Trees, 1984, Monterey, Calif.: Wadsworth and Brooks
8) Koza, Genetic Programming: On the Programming of Computers by Means of natural Selection, 1992, Cambridge, Mass.: MIT Press,
9) Nibet et al. Handbook of Statistical Analysis and Data Mining Applications, 1009: Academic Press
10) Liu and Setiono, Proc. IEEE 7^{th} International Conference on Tools with Artificial Intelligence, 388-391, 1995

1. A method for obtaining prognostic information of a subject determined to have a cancer, the method comprising testing a sample of the cancer to determine whether the subject's cancer has an epithelial phenotype to determine the expression level of three or more of tumor growth factor (TGF)-alpha, miR-200 family members, miR-200 family targets, p63 and CDH-1 as compared to a reference level, wherein:
a) a higher expression level of tumor growth factor (TGF)-alpha as compared to a reference level thereof;
b) a higher expression level of one or more miR-200 family members as compared to a reference level thereof;
c) a lower expression level of one or more miR-200 family targets as compared to a reference level thereof;
d) a higher expression level of p63 as compared to a reference level thereof; and
e) a higher expression level of CDH-1 as compared to a reference level thereof;
indicates an epithelial phenotype and a poor prognosis.

2. The method of claim 1, wherein the epithelial phenotype is determined by an expression profile comprising a) and b) or an expression profile comprising four or all of a)-e).

3. (canceled)

4. The method of claim 1, wherein if the subject's cancer has:
a) an expression level of tumor growth factor (TGF)-alpha not higher than a reference level thereof;
b) an expression level of one or more miR-200 family members not higher than a reference level thereof;
c) an expression level of one or more miR-200 family targets not lower than a reference level thereof;
d) an expression level of p63 not higher than a reference level thereof; and/or
e) an higher expression level of CDH-1 not higher than a reference level thereof;
then such is indicative of a favorable prognosis.

5. The method of claim 1, wherein the one or more miR-200 family members are miR-200b, mir-200c, miR-205, miR-429 and/or miR-141; or wherein the one or more miR-200 family targets are Zinc finger E-box binding homeobox 1 (Zeb-1), Zinc finger E-box binding homeobox 2 (Zeb-2), Zinc figure protein 532 (ZNF532) a-d, ZNF532a&b, and/or ERBB receptor feedback inhibitor 1 (ERRFI-1).

6. (canceled)

7. The method of claim 1, wherein the method comprises using a predictive analytic to generate a prognosis.

8. The method of claim 7, wherein the predictive analytic is neural networks, support vector machines, decision trees, classification and regression trees (CART), or genetic programming.

9. (canceled)

10. The method of claim 7, wherein the predictive analytic comprise one or more rules of:
i) if the subject's cancer has a miR-200b expression level not higher than a reference level thereof, then such is indicative of a favorable prognosis;
ii) if the subject's cancer has a miR-200b expression level higher than a reference level thereof, a TGF-alpha expression level not higher than a reference level thereof, a CDH-1 expression level not higher than a reference level thereof, then such is indicative of a favorable prognosis;
iii) if the subject's cancer has a miR-200b expression level higher than a reference level thereof, a TGF-alpha expression level not higher than a reference level thereof, a CDH-1 expression level higher than a reference level thereof, and a ZNF-532 expression level lower than a reference level thereof, then such is indicative of a poor prognosis;
iv) if the subject's cancer has a miR-200b expression level higher than a reference level thereof, a TGF-alpha expression level not higher than a reference level thereof, a CDH-1 expression level higher than a reference level thereof, and a ZNF-532 expression level not lower than a reference level thereof, then such is indicative of a favorable prognosis;
v) if the subject's cancer has a miR-200b expression level higher than a reference level thereof, a TGF-alpha expression level higher than a reference level thereof, a Zeb-1 expression level lower than a reference level thereof, then such is indicative of a poor prognosis; and
vi) if the subject's cancer has a miR-200b expression level higher than a reference level thereof, a TGF-alpha expression level higher than a reference level thereof, a Zeb-1 expression level not lower than a reference level thereof, then such is indicative of a favorable prognosis.

11. The method of claim 1, wherein the subject is determined to have a cancer of bladder, brain, lung, liver, spleen, kidney, lymph node, small intestine, pancreas, blood cells, colon, stomach, breast, endometrium, prostate, testicle, ovary, skin, head and neck, esophagus, bone marrow or blood.

12. (canceled)

13. The method of claim 1, wherein the method comprises obtaining a sample of the subject's cancer.

14. (canceled)

15. The method of claim 1, wherein the method comprises testing mRNA expression of the subject's cancer.

16. The method of claim 1, wherein the method comprises testing protein expression of the subject's cancer.

17. The method of claim 1, wherein the method comprises analyzing a predetermined expression profile of the subject's cancer.

18. The method of claim 15, wherein the mRNA expression is tested using Northern blotting, quantitative real-time PCR (RT-PCR), nuclease protection, an in situ hybridization assay, a chip-based expression platform, invader RNA assay platform or b-DNA detection platform.

19. (canceled)

20. The method of claim 16, wherein the protein expression is tested using an enzyme-linked immunosorbent assay (ELISA), an immunoassay, a radioimmunoassay (RIA), an immunoradiometric assay, a fluoroimmunoassay, a chemiluminescent assay, a bioluminescent assay, a gel electrophoresis, a Western blot analysis, immunohistochemistry or an expression array.

21. The method of claim 1, further comprising recording the prognostic information in a tangible medium.

22. The method of claim 1, further comprising reporting the prognostic information to the subject, a health care payer, a physician, an insurance agent, or an electronic system.

23. The method of claim 1, wherein the poor prognosis indicates a lower chance of survival as compared with a reference survival level; a higher chance of cancer progression as compared with a reference level thereof or wherein the poor prognosis indicates a poor clinical outcome after a standard therapy.

24-25. (canceled)

26. The method of claim 1, further defined as a method of developing a treatment plan for a subject determined to have a cancer comprising:
a) determining whether the subject's cancer has an epithelial phenotype, wherein if the subject's cancer has an epithelial phenotype, the subject is more likely to exhibit a poor response to one or more conventional cancer therapy and/or a favorable response to a epidermal growth factor receptor (EGFR)-directed therapy; and
b) developing the treatment plan.

27. The method of claim 26, wherein the one or more conventional cancer therapy comprise chemotherapy, radiation therapy, and/or surgery.

28. The method of claim 26, further comprising treating the subject with EGFR-directed therapy if the subject's cancer is determined to have an epithelial phenotype.

29. The method of claim 26, further comprising treating the subject with one or more conventional cancer therapy if the subject's cancer is determined not to have an epithelial phenotype.

30. (canceled)

31. A tangible, computer-readable medium comprising an expression profile of a patient's cancer, wherein the expression profile exhibits expression level of two or more of:
a) TGF-alpha;
b) one or more miR-200 family members;
c) one or more miR-200 family targets;
d) p63; and
d) E-cadherin.

32-34. (canceled)

35. A method of treating the subject having a cancer comprising:
(a) selecting a subject previously determined to have a cancer with an epithelial phenotype in accordance with claim 1; and
(b) administering an EGFR-directed therapy to the selected subject.
